(19) United States
(12) Patent Application Publication
(10) Pub. No.: US 2023/0157299 A1
(43) Pub. Date: May 25, 2023

(54) DISPENSABLE NANOPARTICLE BASED COMPOSITION FOR DISINFECTION

(71) Applicants: UNIVERSITY OF CENTRAL FLORIDA RESEARCH FOUNDATION, INC., Orlando, FL (US); Kismet Technologies LLC, Winter Park, FL (US)

(72) Inventors: Christina Hartsell DRAKE, Winter Park, FL (US); Sudipta SEAL, Orlando, FL (US); Craig NEAL, Orlando, FL (US)

(21) Appl. No.: 17/921,056

(22) PCT Filed: Apr. 30, 2021
(86) PCT No.: PCT/US2021/030221

§ 371 Date: Oct. 24, 2022

Related U.S. Application Data
(60) Provisional application No. 63/018,174, filed on Apr. 30, 2020.

(51) Int. Cl. A01N 59/16; A01P 1/00; A61L 2/18; B82Y 30/00; B82Y 40/00

(52) U.S. Cl. CPC A01N 59/16; A01P 1/00; A61L 2/18; B82Y 30/00; B82Y 40/00; A61L 2101/02

(57) ABSTRACT

Disclosed herein are rapid and residual disinfectant compositions that include a metal-associated cerium oxide nanoparticles. Also disclosed are methods of making disinfectant compositions. Films comprising the disclosed compositions are also disclosed as well as methods of disinfecting a surface.

### BACKGROUND

COVID-19 has brought worldwide challenges to humans due to the ease of transmission of the coronavirus. Transmission is believed to occur primarily via respiratory droplets produced by an infected person as well as by contact with a surface where a droplet containing the SARS-CoV-2 virus exists.[1] Early studies have shown that these viruses can live between 2-3 days on most common types of surfaces.[2] Most known available disinfectants, while able to neutralize many types of viruses, usually require a reaction time on the order of 30 seconds to 10 minutes.[3] This can cause issues when trying to disinfect surfaces where disinfecting at those time scales is not practical. Additionally, current disinfectants require constant reapplication in high contact areas because they do not provide residual protection against both viruses and bacteria.

### BRIEF DESCRIPTION OF THE DRAWINGS

FIG. 1 shows RAD compositions at application and post application. Ceria Nanoparticles (CNP) mechanisms of virus deactivation shown in bottom right box.

FIG. 2A shows x-ray photoelectron spectroscopy (XPS) survey scan of silver-modified cerium oxide nanoparticles (AgCNPs), FIG. 2B shows unique multiplet cerium signatures used to quantify Ce³⁺/Ce⁴⁺ ratio, FIG. 2C shows silver peaks detailing silver chemical environment in AgCNPs, FIG. 2D is a hrTEM of siver-modified CNP, FIG. 2E is x-ray diffraction of pure phase CNPs.

FIG. 3 are flow charts showing the syntheses for AgCNP1 and 2.

FIG. 4 is a model of the syntheses for AgCNP1 and 2.

FIG. 5 shows material characterization of AgCNP1 and 2. FIG. 5A is a TEM image of AgCNP1 showing the spherical particles (with the size of 20 nm) enriched with Ag nanoparticle (with the size of 2-5 nm). FIG. 5B is a TEM micrograph of AgCNP2 showing the agglomerated CeO2 particles designed with various sizes of Ag nanoparticles (5 to 20 nm). Tafel analysis for AgCNP 1 and 2 FIG. 5C showing unique corrosion potentials for each formulation (465.386 and 217.374 mV, respectively). FIG. 5D is a Nyquist represation of AgCNP1 and 2 from 10 Hz to 100 kHz.

FIG. 6 shows in situ measurements of AgCNP-Virus interactions via impedance spectroscopy. FIGS. 6A-C show the incubation of AgCNP1 with OC43, enveloped coronavirus; FIGS. 6D-F are related to AgCNP2 incubation with non-enveloped rhinovirus measured at regular time intervals of 30 minutes (total 2 and 4 hours for Rhinovirus and OC43 virus incubations, respectively).

FIG. 7 is the Electrochemical model of in situ AgCNP-virus interactions.

FIG. 8 is the physical model of virus/nanoparticle interaction: Liposome/Xanthine:Xanthine Oxidase. FIG. 8A is the fitted electrochemical impedance spectra, FIG. 8B shows the equivalent circuit, and FIG. 8C is the fitted circuit element values.

FIG. 9 is a graph showing AgCNP2 dried on a slide efficacy against RV14.

FIG. 10 are graphs showing the repeat efficacy of the AgCNPs.

### DETAILED DESCRIPTION

Disclosed herein is a Rapid and Residual Acting Disinfectant (RAD) composition, (e.g. nanoRAD) to curb the transmission of SARS-CoV-2, and other pathogens, via contact with surfaces. The disclosed approach employs a select medium containing fast-response metal-associated cerium oxide nanoparticles where the oxidizing response/mechanism is engineered to perform several ‘disinfectant’ reactions in parallel. The first is an oxidation reaction involving the virus spike glycoproteins which inhibits virus-host cell interaction, thus, inactivating infectivity. The second mechanism is membrane peroxidation of the virus envelope to induce lysis; thereby, rendering it ineffective. Each mechanism of disinfection can be accomplished via cerium oxide surface reactions. These mechanisms are self-regenerating since the nanoparticles are not used up in the disinfection process, allowing nanoRAD to have residual disinfection capabilities. In a further embodiment, the particles may be made more efficacious through incorporation of silver: leading to further generation of free radicals in application. Doping of nanoceria with fluorine, or similar chemistry, may be done to decrease the reaction rate of the first two mechanisms, to well below 30 seconds. The combination of disinfecting mechanisms, working together, will reduce the overall rate event further, allowing for rapid disinfection by multiple concurrent routes, and dry disinfecting potency at concentrations that are safe for contact.

According to one embodiment, disclosed is a dispensable composition including a metal-associated cerium oxide nanoparticles (mCNP) and an excipient. The metal associated with the cerium oxide nanoparticles may include but is not limited to silver, gold, ruthenium, vanadanium, copper, titanium, nickel, platinum, titanium, tin and iron. In a specific example, the metal is silver and comprises 10% or less of the weight of the particle. In some embodiments, the excipient is selected from the group consisting of water, chloroform, methylene chloride, acetone, methyl ethyl ketone, cyclohexane, ethyl acetate, diethyl ether, lower alcohols, lower diols, THF, DMSO, or DMF. The mCNPs may be further doped with fluorine.

In other embodiments, disclosed is a method of producing mCNPs. Where the metal is silver, the AgCNPs are produced via a method comprising dissolving cerium and silver precursor salts such as cerium and silver nitrates; oxidizing the dissolved cerium and silver precursor salts via admixture with peroxide; and precipitating nanoparticles by subjecting the admixture with ammonium hydroxide. Alternatively, the AgCNPs are produced via a method comprising (i) dissolving cerium and silver precursor salts such as cerium and silver nitrates; (ii) oxidizing and precipitating the dissolved cerium and silver precursor salts via admixture with ammonium hydroxide; (iii) wash and resuspend precipitated nanoparticles in water; (iv) subject the resuspended nanoparticles with hydrogen peroxide; and (v) washing the nanoparticles from step (iv) to remove ionized silver.

In a further embodiment, disclosed is a method of disinfecting a surface by dispensing a dispensable composition embodiment onto the surface. These and other embodiments are further described below.

### Definitions

Unless specifically stated or obvious from context, as used herein, the term “about” is understood as within a range of normal tolerance in the art, for example within 2 standard deviations of the mean. About can be understood as within 30%, 25%, 20%, 15%, 10%, 9%, 8%, 7%, 6%, 5%, 4%, 3%, 2%, 1%, 0.5%, 0.1%, 0.05%, or 0.01 % of the stated value. Unless otherwise clear from context, all numerical values provided herein are modified by the term about.

The terms “disinfection” or “disinfect” as used herein refers to a reduction or elimination of pathogenic microorganisms on surfaces including bacteria and viruses. The term “residual disinfection” as used herein refers to any sprayed disinfectant capable of disinfecting a surface for at least 24 hours in dry form. Residual disinfectants that last up to 24 hours disinfect 3log reduction of viral load and 5log reduction of bacterial load in under 10 minutes. Residual disinfectants (sprayed or applied by other means) that persist longer than a day disinfect at 3log reduction viral load and 3log reduction bacterial load within 2 hours.

The term “rapid disinfection” as used herein refers to near instantaneous elimination of a pathogenic microorganism on surfaces. Rapid disinfectants have a dwell time for disinfection of about 1 minute or less when applied in wet form.

The term “metal-associated cerium oxide nanoparticles”, “metal-associated ceria nanoparticles”, or “mCNPs” refers to cerium oxide nanoparticles doped with or otherwise bound to a metal such as silver, gold, copper, platinum, nickel, iron, titanium, ruthenium, vanadanium and the like. The term mCNPs includes AgCNPs. In an embodiment, the metal-associated cerium oxide nanoparticles comprise a particle size of the range of from 1 nm to 50 nm or from 5 nm to 100 nm or from 5 nm to 25 nm.

The term “nanoRAD” as used herein refers to a disinfectant with cerium oxide nanoparticles associated with a metal such as silver as the active agent and a an excipient. As taught herein, the disclosed nanoRAD compositions may include excipients such as organic acid, surfactant, drying agent and/or polymer, among others.

The term “dispense”, as used herein, refers generally to the ejection of a composition from a container or dispensing system. The dispensing may be, for example, accomplished by using a air exchange pump, opening, or the like. There is no limitation on the amount or manner in which a composition is dispensed. In certain embodiments a composition may be dispensed as a fine mist that resembles an aerosolized spray, which may be accomplished by using, for example, a nozzle or atomizer. In other embodiments the composition may be dispensed as a single stream of liquid, as drops, under high or low pressure, and so forth. Any form of dispensing that meets the needs of a particular circumstance may be utilized in embodiments of the present invention.

The term “pump”, as used herein, refers to a device that is capable of dispensing a composition that is located within a container. The pump may be an “air-exchange” pump that functions by injecting air or the like into the container. The injected air then displaces and dispenses some or all of the composition within the container. The amount of composition dispensed depends on the amount of air injected and amount of composition within the container. More specifically, a pump may inject air into a container and dispense the composition out of a nozzle or other opening.

The term “predominant 4+ surface charge” refers to the concentration of cerium ions on the surface and means that the [Ce3+]:[Ce4+] ratio on the surface of the cerium oxide nanoparticle is less than 50%. In a specific example, cerium oxide nanoparticles having a predominant 4+ surface charge have a [Ce3+]:[Ce4+] ratio that is 40% or less.

The term “predominant 3+ surface charge” means that the [Ce3+]:[Ce4+] ratio on the surface of the cerium oxide nanoparticle is greater than 50%. In a specific example, the [Ce3+]:[Ce4+] ratio is greater than 60%.

The term “wet chemical synthesis” refers to a method of making CNPs that involves dissolving a cerium precursor salt in water followed by addition of hydrogen peroxide. In a specific example, the CNPs are stabilized over a predetermined time period, typically at least 15-30 days.

### Overview

Current disinfectant sprays only disinfect at the time of application. After application, the disclosed RAD compositions have the unique ability to create a temporary, continually disinfecting film left behind on the surface to which it was applied. The persistent, disinfectant activity is due to the regenerative (catalytic) properties of Ceria Nanoparticles (CNP) nano-surface reaction sites which allow for continued disinfection of a surface when new viruses or bacteria come into contact with it. For surfaces where a permanent disinfectant film is not easily applied, this presents an attractive solution. RAD compositions are a solution that can curb transmission of COVID 19 and Hospital Acquired Infections (HAIs) via contact with surfaces in a manner that is not currently available and is unique as a disinfectant spray and temporary film.

With the rise of COVID-19, many businesses and governments have struggled with how to allow people to be in public or communal spaces in a way that mitigates spreading of the coronavirus. In India, a walk-through sprinkler system has been used to spray disinfectant directly on market shoppers as they enter the marketplace.[4] Because of the high transmissibility of the coronavirus, many have scrambled to find a solution to curb transmission even when the benefits are not clear.

Coronavirus, like many respiratory viruses, is spread through respiratory droplets. This means while people are present in an area, sneezing, talking and coughing have the ability to deposit respiratory droplets onto surfaces. On a normal surface, with the use of commercially available disinfectant sprays, these droplets would retain any viruses already embedded within them in a stable form until a disinfectant spray is applied, or after a time period ( potentially as long as 2 to 3 days) has passed. Permanent anti-viral films are being researched to help curb the transmission of SARS-CoV-2. Permanent films have adhesion requirements specific to the surface it is applied to prevent delamination. Further, these films largely aim to prevent wetting of a surface, as an indirect measure against virus transmission, and do not directly inactivate virus species. RAD compositions would have the ability to keep surfaces disinfected for longer periods of time than what is currently available. Permanent disinfectant films can be difficult to retrofit to existing surfaces and may require replacement/modification of parts or materials to provide their benefit. RAD compositions, when commercially available, will combine the benefits of commercially available sprays and films by providing the acute disinfecting power of a spray that has little persistence with some of the benefits of a permanent film.

The Centers for Disease Control (CDC) has guidelines for surface disinfection in childcare facilities through the group National Resource Center for Health and Safety in Child Care and Early Education.[6] The recommended disinfection schedule includes guidance for before use, after use, and daily (at the end of each day), Table 1. It should be noted that this recommended schedule was linked from the CDC website on daycare facility guidance for COVID-19.[7] Chosen for this table were often touched surfaces that could contribute to the spread of coronavirus. Many of these are only recommended to be cleaned at the end of the day. Given the highly contagious nature of SARS-CoV-2, and the fact that many people are asymptomatic but carriers of the virus, these cleaning measures would not be sufficient. They present opportunities for someone to sneeze, cough, or talk, near a surface and deposit respiratory droplets while never actually physically contacting the surface. However, application of RAD compositions to extend the disinfection time after application would make this disinfection schedule more reliable in preventing virus transmission via surfaces.

TABLE 1

Routine Schedule for Cleaning, Sanitizing and Disinfecting (adapted from [6])

| Areas | Before Each Use | After Each Use | Daily (At the End of Day) | Weekly | Monthly | Comments |
|---|---|---|---|---|---|---|
| Door and cabinet handles | | | Clean, Disinfect | | | |
| Floors | | | Clean | | | Sweep then mop |
| Handwashing sinks and faucets | | | Clean, Disinfect | | | |
| Computer Keyboards | | Clean, sanitize | | | | Use sanitizing wipes |
| Phone receivers | | | Clean | | | |
| Toilets | | | Clean, Disinfect | | | |
| Toilet area floor | | | Clean, Disinfect | | | Damp mop with floor disinfectant |

. The disclosed RAD compositions, unlike other available surface disinfectants, provides a capability that is not currently available by surface disinfectants: a temporary, continually disinfecting film. For consumers in charge of places for high risk of transmission of the coronavirus, this feature will make the RAD compositions an attractive alternative solution

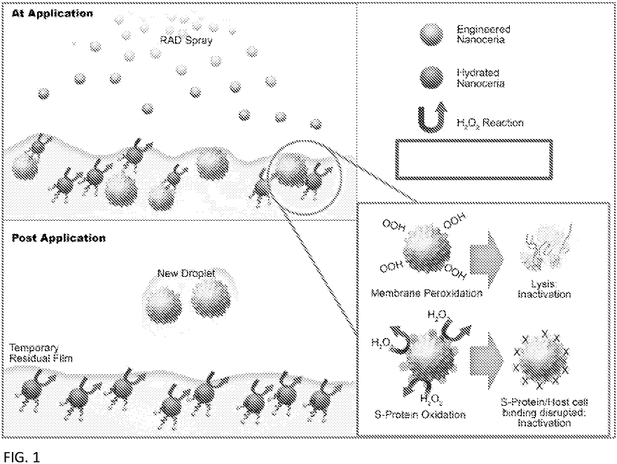

In one embodiment, provided is a Rapid Acting Disinfectant (RAD) Spray that curbs the transmission of viruses (e.g. SARS-CoV-2) via contact with contaminated surfaces. The RAD spray employs a select medium containing fast-response doped CNPs where the oxidizing response is engineered to perform several disinfectant mechanisms in parallel (Table 2). FIG. 1 shows a concept of operation for how the RAD compositions works to act against respiratory viruses like the coronavirus Reactive oxygen species (ROS) generation, is one of the mechanisms that is used along with other direct CNP surface reaction mechanisms (membrane peroxidation and S-protein oxidation) to improve the rate of disinfection as well as the disinfecting efficiency of each individual CNP.[14] The combination of disinfecting mechanisms, working together, improves the overall disinfectant rate, allowing for rapid and potent disinfection by multiple concurrent routes. After application, the disclosed RAD compositions have the unique ability to create a temporary, continually disinfecting film left behind on the surface to which it was applied. CNPs have regenerative properties that allow for continued disinfection of a surface when new viruses from respiratory droplets or physical transmission encounter it. For surfaces where a permanent disinfectant film is not easily applied, this presents an attractive solution to allow for application to multiple types of surfaces regardless of the surfaces’ ability to adhere to a film. In a specific example the RAD composition is a solution that can curb transmission of COVID 19 and other pathogens via contact with surfaces in a manner that is not currently available and is unique as both a disinfectant spray and temporary film. These mechanisms are discussed in greater detail herein.

TABLE 2

NanoRAD is a rapid acting, residual disinfectant spray that continues to safely disinfect for days after it has been initially applied and performed disinfection on a surface

| | | |
|---|---|---|
| Feature | Advantage | Benefit |
| Residual (Self) Disinfection | Higher client throughput | Decreased business downtime due to disinfection |
| Residual (Self) Disinfection | More labor spent with clients or customers | Decreased manpower and resources from business dedicated to disinfection |
| Rapid Disinfection | Faster disinfection process | Decreased disinfection turn-around time |
| Rapid Sprayed Disinfectant | Ease of application | Decreased confusion on dwell time requirements to achieve disinfection |
| Low Chemical Irritancy | Lower PPE requirements | Decreased wellness concerns about dispensing of disinfectant |

Currently, CNPs have been used experimentally in vitro as broad-spectrum antiviral agents. They are used as an alternative approach to prevent viral infections due to their unique chemical (e.g. enhanced catalytic activity) properties. It is hypothesized that when NPs become hydrated by bio-fluids (e.g. respiratory droplets), surface redox reactions produce ROS and a concomitant oxidative stress inducing lipid peroxidation of the viral envelope, affecting stability of the virus causing oxidation of surface receptor proteins, thereby inactivating the virus to infectivity (i.e. by modifying the receptor to preclude host cell-virus interaction)

Different types of nanoparticles have been proven as antiviral agents such as gold, silver, and ceria. Among these, CNPs have minimal or no toxicity towards normo-typic cells and modulate redox related cell processes towards cell survival or death, and demonstrate unique catalytic activity towards oxygen metabolic species, based on synthesis protocol. Ceria can exist in two forms: 1) as Ce₂O₃ with hexagonal [27] and 2) as CeO₂ with a cubic fluorite lattice. This gives nanoceria with properties: oxygen storage and release, catalysis [27, 28] and solar/fuel cells.[29]

In the case of CNPs, creation of an oxygen vacancy leads to localization of two electrons over 4f states. [27, 30, 31] This results in reduction of two coordinated cerium cations (from Ce⁴⁺ to Ce³⁺)/oxygen with a thermodynamically stable structure. [27, 31] In addition, the surface area available and the orientation of crystallographic planes in nanoceria highly regulate the catalytic property at nanoscale level. It has been previously demonstrated that the (100) family of planes [32] of nanoceria exhibit the highest reactivity, among the most atomically dense crystal planes, due to their relatively high inter-atomic spacings. [33] This was previously illustrated by changing the morphology of nanoceria particles, which can be controlled by changing the synthetic method of preparation, and determining Madelung energies at varied crystal planes [34].

These oxygen vacancies become the sites for the catalytic activity and varies with particle size.[35] CNPs have diverse enzyme- mimetic activity depending on their surface chemistry. The catalase mimetic activity is high due to the presence of +4 surface oxidation state while superoxide dismutase activity increases with more Ce³⁺. [36, 37] Also, these mixed-valence states in CNPs (Ce³⁺ to Ce⁴) have the ability to switch between oxidation states inside the crystal system. When switching its valence state, CNPs can scavenge reactive oxygen species (ROS) and reactive nitrogen species (RNS). In a biological system, important biological and environmental reactions take place by pro-oxidants and antioxidants. The pro-oxidants induce oxidative stress (that can cause virus damage) either by producing hydroxyl radical (OH-), hydrogen peroxide (H₂O₂), and the superoxide anion (O_{2‾}). Catalytic CNP has been used to reduce reactive oxygen species in various organs of the human body under normal and cancerous conditions through redox reactions. [16, 38-40]

CNPs are used as an antimicrobial [41] and antiviral agent.[42] Nanoceria acts as an antibiotic agent by acting directly on bacterial structure or indirectly through chemical modification. CNPs can interact directly with a bacterial cell wall leading to cell wall destabilizing and lysis. Alternatively, particles can function indirectly; reacting with intra-cellular chemical species and components. Each mechanism leads to bacterial cell death. The positive charge on CNPs at physiological pH’s leads to antimicrobial activity against the bacterial species based on these mechanisms, mediated by initial membrane adherence. [43, 44] In the case of a virus, the geometry, and the surface charge of the CNPs play an important role to act as an antiviral agent. The unique biochemical properties and an intercellular cascade of virus-motivated biochemical reactions can be modified by attachment of a CNP to the virus surface prior to cell permeation/virus uptake. Lozovski et al demonstrated that a narrow, small size CNP distribution has the most significant effect against DNA- and RNA-containing viruses.[42, 45] This was due to the local effect of released ions eliciting phosphatase-mimetic activities, as well as interfering with calcium-dependent membrane processes. Additionally, these ionic species were demonstrated to modulate metabolic processes, especially at or near mitochondria (e.g. electron transport chain events).[46] CNPs easily attach to phosphate groups leading to inorganic, insoluble cerium phosphate. [47] Further, CNPs have been demonstrated to accelerated the cleavage of highly resistant phosphodiester bonds in nucleic acids.[46] When a CNP interacts with cell surface proteins it leads to cell surface property changes. These can include membrane colloidal property and its fluidity, thus affecting the ability of the virus to enter into living cells. Specially designed nanoceria, with or without Ag dopant, is a candidate for comprehensive antiviral therapy and deactivation of surface contamination created by emerging COVID-19 and other viruses and pathogens.

### Description of Embodiments

The present disclosure describes cerium oxide nanoparticles doped with or otherwise bound to a metal such as silver, gold, copper, platinum, nickel, iron, titanium, ruthenium, vanadanium and the like. Use of metal and metal oxide nanomaterials have been studied in a variety of anti-bacterial/viral applications, with a broader basis for pathogen toxicity. Transition metal-based materials have shown exceptional broad-spectrum anti-bacterial activity as well as anti-viral efficacy.

The mCNP may be spherical, rod-shaped, star-shaped, or polygonal. In a preferred embodiment, the mCNP are spherically-shaped, meaning that they more or less approximate the shape of a sphere. Preferably, the average diameter of the spherically-shaped mCNP is about 24 nm or less, about 20 nm to about 24 nm or about 3 nm to about 5 nm. In a certain embodiment, the spherically-shaped cerium oxide nanoparticles have an average diameter of 3 nm to 5 nm as measured by transmission electron microscopy. In embodiments in which the mCNP are not spherically shaped, it is preferred that the average dimension between two opposing sides of the nanoparticles is 24 nm or less.

The mCNP have a cerium oxide core with an external surface. The surface is characterized according to the percentage of Ce(3+) relative to Ce(4+) ions thereon. Although the amount is not intended to be limiting, when used in methods of the invention, some preferred ranges of Ce(3+):Ce(4+) percentages are: about 80%:20% to about 20%:80%, about 75%:25% to about 25%:75%, about 60%:40% to about 25%:75%, or about 57%:43% to about 27%:73%. In certain embodiments, the percentage of Ce(3+) relative to Ce(4+) is >50% Ce(3+).

### Silver Associated Cerium Oxide Nanoparticles

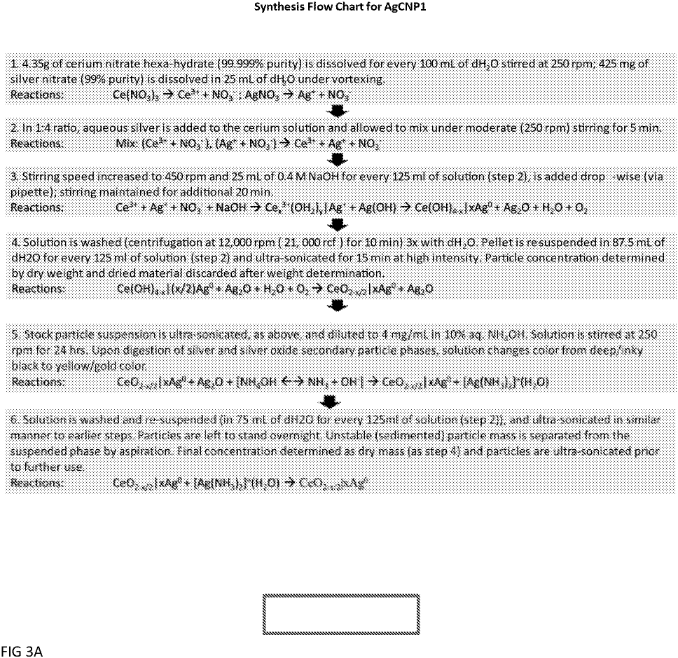

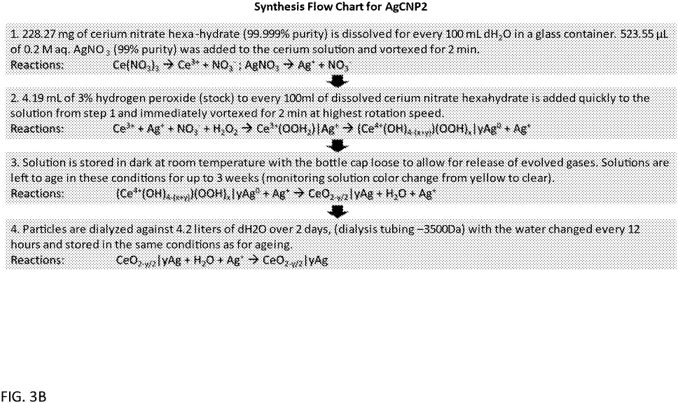

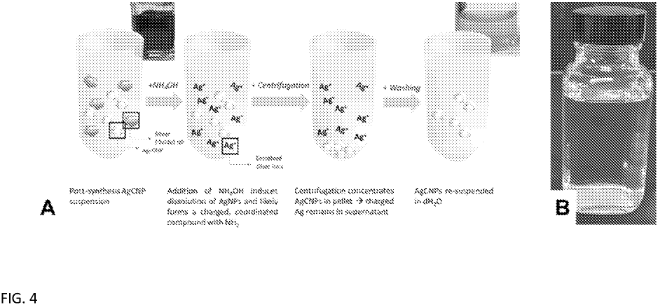

The present disclosure includes the two different types of nanoparticles AgCNP1 and AgCNP2. In certain embodiments, there is a combination of the two since they seem to have slightly different modes of action. Silver modified cerium oxide formulations (AgCNPs) are synthesized in two unique formulations (AgCNP1, AgCNP2) each utilizing different chemical reactions specific to aqueous silver. AgCNP1 is synthesized via a previously developed, two step procedure (FIG. 3A, FIG. 4) that can be scaled to a large or small process. Briefly, a solution containing AgCNP-like, silver-modified nanoceria, and silver secondary phases are formed via an alkaline-forced hydrolysis reaction. The product materials are washed with dH₂O and subsequently treated with ammonium hydroxide. Ammonium hydroxide functions as an etchant as well as a phase transfer complex: mediating the solubilization/stabilization of dissolved silver ions in the aqueous phase. In particular, the reaction results in the formation of Tollen’s reagent (Ag[(NH₃)₂OH]_{aq}). The resulting single particle solution is then washed with dH₂O to remove excess base and counter/spectator ions. AgCNP2 utilizes the stability of silver ions towards oxidation by hydrogen peroxide (FIG. 3B). Specifically, dissolution of cerium and silver nitrates followed by addition of hydrogen peroxide leads to the selective oxidation of cerium ions over silver and the evolution of metallic silver phases on the ceria surface. The unique synthesis conditions of these particles suggest a potentially disparate particle character. In certain embodiments the synthesis can be scaled to large or small processes.

Example of small-scale process of AgCNP2:
1. 109 mg of cerium nitrate hexa-hydrate (99.999% purity) is dissolved in 47.75 mL dH₂O in a 50 ml square glass bottom
2. 250 µL of 0.2 M aq. AgNO₃ (99% purity) is added to the cerium solution above with the solution vortexed for 2 minutes: Machine: Vortexer
3. From here, 2 mL of 3% hydrogen peroxide (stock) is added quickly to the above solution followed by immediate vortexing for 2 minutes at highest rotation speed (in vortexer machine)
4. Solution is stored in dark condition at room temperature with the bottle (50 mL square bottom glass) cap loose to allow for release of evolved gases; solutions are left to age in these conditions for up to 3 weeks (monitoring solution color change from yellow to clear) to create 50 ml total volume of the solution
5. Particles are then dialyzed against 2 liters of dH₂O over 2 days, (dialysis Tubing) with the water changed every 12 hours and stored in the same conditions as for ageing.

The two unique formulations of cerium oxide nanoparticles are produced with surfaces modified by silver nano-phases. Materials characterization shows that the silver components in each formulation are unique from each other and decorate the ceria surface as many small nanocrystals (AgCNP1) or as a Janus-type two-phase construct (AgCNP2). Preferably, the average diameter of AgCNP1 is about 20 to 24 nm, and the average diameter of AgCNP2 is about 3 to 5 nm. Each synthesis further possesses unique mixed valency with AgCNP2 possessing a significantly greater fraction of Ce³⁺ states relative to Ce⁴⁺ over AgCNP2. The distinct valence characters, along with incorporation of chemically active silver phases, lead to high catalytic activities for each formulation. AgCNP2 possesses high superoxide dismutase activity, while AgCNP1 possesses both catalase and superoxide dismutase-like enzyme-mimetic activities, ascribed to the catalase activity of ceria and the superoxide dismutase activity from silver phases. Further, electrochemical analysis demonstrates that silver incorporated in each formulation is substantially more stable to redox-mediated degradation than pure silver phases: promoting an increased lifetime in catalytic applications. Use of each formulation in effecting anti-viral properties showed a specific activity for each formulation: with, among the virus species tested, AgCNP1 showing substantial activity towards OC43 coronavirus and AgCNP2 active against RV14 rhinovirus. In situ electrochemical impedance spectra collected for each virus/particle system over the respective incubation periods mirrored the unique interactions observed for each pairing. Equivalent circuit fittings for each, along with developed model/test systems (use of analog virus-like particles, model protein, radical oxygen species generating enzyme/substrate systems), showed the modes of action for the pairings in effecting anti-viral responses. The results of these investigations assign a dominate physical interaction-based mechanism for OC43/AgCNP1 while an oxidative, chemical interaction is determined for RV14/AgCNP2.

Although the amount is not intended to be limiting, when used in methods of the invention, some preferred amounts of silver percentages associated with the AgCNPs are about 6% to about 10%, or less.

### Implementations of Compositions

In one embodiment, provided is a dispensable composition comprising mCNPs (e.g. AgCNPs) and an excipient. Examples of excipients include solvents such as but are not limited to, water or water-based (aqueous) solutions in which water is at least the main component, lower alcohols (C6 or lower), lower diols (C6 or lower), THF, DMSO, DMF, etc. They can be used alone or as mixtures of various components with water. Examples that do not constitute limitation of nonaqueous carriers or mixtures thereof are chloroform, methylene chloride, acetone, methyl ethyl ketone, cyclohexane, ethyl acetate, diethyl ether, lower alcohols (C4 or less), lower diols (C4 or less), THF, DMSO and DMF.

The dispensable composition may also comprise a fragrance. Examples of fragrance include, but are not limited to, emon oil, orange oil, bergamot oil, ylang ylang oil, patchouli oil, citronella oil, lemongrass oil, boad rose oil, clove oil, eucalyptus oil, cedar oil, lavender oil, Natural fragrances such as sandalwood oil, vetiver oil, geranium oil, labdanum oil, peppermint oil, rose oil, jasmine oil, litz accubeba oil; hydrocarbon-based fragrances (eg limonene, α-pinene, camphene, p-cymene, phen Chen, etc.), ether perfumes (for example, 1,8-cineole, rose oxide, cedrol methyl ether (cedlum bar), p-cresyl methyl ether, isoamylphenyl ethyl ether, 4-phenyl-2,4,6-trimethyl- 1,3-dioxane, anethole, etc.), S Perfume (for example, ethyl acetate, ethyl propionate, methyl butyrate, ethyl isobutyrate, ethyl butyrate, butyl acetate, ethyl 2-methylbutyrate, isoamyl acetate, ethyl 2-methylpentanoate (manzanate) , Hexyl acetate, allyl hexanoate, tricyclodecenyl propionate (VERTOPRO; fluorocyclene), allyl heptanoate, isobornyl acetate, linalyl acetate, citronellyl acetate, 2-ter-butylcyclohexyl acetate (narcidol) Etc.), alcoholic fragrances (eg, linanol, 3-octanol, 2,6-dimethyl-heptanol, 10-undecenol, geraniol, nerol, citronellol, rosinol, mill Senol, tetrahydrolinalol, thymol, terpineol, cedrol, 2,4-dimethyl-3-cyclohexane-1-methanol, 4-isopropylcyclohexanol, nerolidol, 9-decenol, cis-3-hexenol, trans-2-hexenol, eugenol, etc.), Aldehyde perfume (for example, citronellal, para aldehyde, benzaldehyde, aldehyde C-6, aldehyde C-7, aldehyde C-8, aldehyde C-9, aldehyde C-10, tripral, p-ethyldimethylhydrocinnamic aldehyde) Synthetic fragrances such as (florazone), 2-tridecenal, aldehyde C11, etc.) or blended fragrances blended with these.

According to a further embodiment, a substrate may be coated with a film of metal-associated cerium oxide nanoparticles as taught herein. The substrate may take the form of any surface upon which human contact is made or human expired droplets are commonly disposed such as tissues, tissue paper, countertops, HVAC filters, air cleaning devices, electric fans, refrigerators, microwave ovens, dish washer/driers, rice cookers, pots, pot lids, IH heaters, washing machines, vacuum cleaners, lighting apparatuses (lamps, apparatus bodies, shades, and the like), sanitary products, toilets, washbowls, mirrors, bathrooms (walls, ceilings, floors, and the like), building materials (interior walls, ceiling materials, floors, exterior walls, and the like), interior products (curtains, carpets, tables, chairs, sofas, shelves, beds, beddings, and the like), glasses, sashes, hand rails, doors, knobs, clothes, filters used for home electric appliances or the like, stationery, kitchen utensils, medical supplies (white coats, masks, gloves, and the like), medical appliances and devices, and materials used inside automobiles, vehicles of trains, aircrafts, boats and ships, and the like. Examples of a substrate material include glass, ceramics, plastic, resin such as acryl, paper, fiber, metal, wood, and the like.

In another embodiment, one may also produce antiviral foams which are used for a number of applications. For example, polyurethane foams are made using a formulation produced by mixing an isocyanate with a polyol (a molecule with three or more hydroxyl groups) a chain extender (a bifunctional hydroxyl molecule), catalysts to promote reaction, surfactant, heat and/or UV stabilizers along with a foaming agent. The foaming agent could be water as it produces carbon dioxide gas when it reacts with the isocyanate. One method of making antiviral foams involves producing metal-associated cerium oxide nanoparticles with a surfactant (using a surfactant compatible with the system or the same which is used in the system) or one of the urethane-forming constituents and adding these to the foam formulation. Another alternative involves producing nanoparticles in an aqueous media, such as by mixing them in water along with the desired surfactant and then adding this aqueous mixture to the foam formulation both as a foaming agent and as an antiviral source.

According to other embodiments, antiviral inks comprising cerium oxide nanoparticles associated with silver or another metal may be formed using techniques known in the art of printing inks. Such inks may be printed using a variety of techniques such as inkjet, flexo, gravure and silk-screening. In some cases, such as in inkjet printing, the size of the functionalized particles should be smaller than about 50 nm. Three dimensional antiviral products (mask material and hard objects commonly touched) may be formed by 3-D printing, where the 3-D printing compositions incorporate the antiviral materials taught herein such as AgCNPs.

### Spray Formulation

The present disclosure also includes spray formulations of nanoRAD. In typical embodiments, the formulations comprise nanoRAD, a drying agent, an organic acid, surfactants, water, and a polymer binder. In certain embodiments, nanoRAD may comprise one or several mCNPs dependent on the disinfectant mechanisms needed. The nanoRAD spray creates a disinfecting film when applied to a substrate. In certain embodiments, nanoRAD is in an amount ranging from about 0.01 to 10% by weight. In certain embodiments, a drying agent, such as ethanol or isopropanol, is in an amount ranging from about 0 to 40% by weight. In certain embodiments, about 0.5 to 2% citric acid, or other organic acids, by weight is provided to the spray formulation. Other drying agents include an alcohol or a mixture of alcohols, for example, ethanol, isopropyl alcohol, n-propyl alcohol, and mixtures thereof; fatty alcohols, including, but not limited to, cetyl alcohol, myristol alcohol, stearyl alcohol, octyl alcohol, decyl alcohol and lauryl alcohol, and mixtures thereof; hexanol, and/or other aliphatic or aromatic alcohol. Organic acids that may be used in the disclosed compositions include, but are not limited to, lactic acid, citric acid, salicylic acid, glycolic acid, mandelic acid, benzoic acid and combinations thereof.

The nanoRAD can also be mixed with surfactants, diluents, and polymer binders which are compatible as selected in accordance with the route of application. Surfactants may act as detergents, wetting agents, emulsifiers, foaming agents, or dispersants. In certain embodiments, surfactants are in an amount ranging from about 0.5 to 3% by weight. Suitable surfactants are for example, lauramine oxide, myristamine oxide, other zwitterionics, tergitol 15-S-15 or other secondary alcohol ethoxylate. In certain embodiments, lauramine oxide is in an amount ranging from about 0.25 to 2% by weight, and tergitol 15-S-15 is in an amount ranging from about 0 to 1% by weight. In certain embodiments, the suitable diluent is water and is in an amount ranging from about 15 to 45% by weight. Polymer binders are used to produce transparent, flexible, oxygen permeable films which adhere to glass, plastics and metals. Suitable polymer binders are for example, Poly(2-ethyl-2-oxazoline) or PVP-Vinyl Acetate copolymers. In certain embodiments PVP-Vinyl Acetate copolymers is in an amount ranging from about 1 to 30% by weight. In certain embodiments, Poly(2-ethyl-2-oxazoline) is in an amount ranging from about 1 to 25% by weight.

Other polymers suitable for use with the disclosed compositions include polyethylene oxide (Polyox) hydrogel polymer, stearyl alcohol, cellulose polymer, cationic hydroxy ethyl cellulose (e.g., Ucare; JR30), hydroxy propyl methyl cellulose, hydroxy propyl cellulose (Klucel), chitosan pyrrolidone carboxylate (Kytamer), behenyl alcohol, zinc stearate, emulsifying waxes, including but not limited to Incroquat and Polawax, an addition polymer of acrylic acid, a resin such as Carbopol® ETD 2020, guar gum, acacia, acrylates/steareth-20 methacrylate copolymer, agar, algin, alginic acid, ammonium acrylate co-polymers, ammonium alginate, ammonium chloride, ammonium sulfate, amylopectin, attapulgite, bentonite, C9-15 alcohols, calcium acetate, calcium alginate, calcium carrageenan, calcium chloride, caprylic alcohol, carbomer 910, carbomer 934, carbomer 934P, carbomer 940, carbomer 941, carboxymethyl hydroxyethyl cellulose, carboxymethyl hydroxypropyl guar, carrageenan, cellulose, cellulose gum, cetearyl alcohol, cetyl alcohol, corn starch, damar, dextrin, dibenzlidine sorbitol, ethylene dihydrogenated tallowamide, ethylene diolamide, ethylene distearamide, gelatin, guar gum, guar hydroxypropyltrimonium chloride, hectorite, hyaluronic acid, hydrated silica, hydroxybutyl methylcellulose, hydroxyethylcellulose, hydroxyethyl ethylcellulose, hydroxyethyl stearamide-MIPA, isocetyl alcohol, isostearyl alcohol, karaya gum, kelp, lauryl alcohol, locust bean gum, magnesium aluminium silicate, magnesium silicate, magnesium trisilicate, methoxy PEG-22/dodecyl glycol copolymer, methylcellulose, microcrystalline cellulose, montmorillonite, myristyl alcohol, oat flour, oleyl alcohol, palm kernel alcohol, pectin, PEG-2M, PEG-5M, polyacrylic acid, polyvinyl alcohol, potassium alginate, potassium aluminium polyacrylate, potassium carrageenan, potassium chloride, potassium sulfate, potato starch, propylene glycol alginate, sodium acrylate/vinyl alcohol copolymer, sodium carboxymethyl dextran, sodium carrageenan, sodium cellulose sulfate, sodium chloride, sodium polymethacrylate, sodium silicoaluminate, sodium sulfate, stearalkonium bentotnite, stearalkonium hectorite, stearyl alcohol, tallow alcohol, TEA-hydrochloride, tragacanth gum, tridecyl alcohol, tromethamine magnesium aluminium silicate, wheat flour, wheat starch, xanthan gum, abietyl alcohol, acrylinoleic acid, aluminum behenate, aluminum caprylate, aluminum dilinoleate, aluminum salts, such as distearate, and aluminum isostearates, beeswax, behenamide, butadiene/acrylonitrile copolymer, C29-70 acid, calcium behenate, calcium stearate, candelilla wax, carnauba, ceresin, cholesterol, cholesterol hydroxystearate, coconut alcohol, copal, diglyceryl stearate malate, dihydroabietyl alcohol, dimethyl lauramine oleate, dodecanoic acid/cetearyl alcohol/glycol copolymer, erucamide, ethylcellulose, glyceryl triacetyl hydroxystearate, glyceryl tri-acetyl ricinolate, glycol dibehenate, glycol di-octanoate, glycol distearate, hexanediol distearate, hydrogenated C6-14 olefin polymers, hydrogenated castor oil, hydrogenated cottonseed oil, hydrogenated lard, hydrogenated menhaden oil, hydrogenated palm kernel glycerides, hydrogenated palm kernel oil, hydrogenated palm oil, hydrogenated polyisobutene, hydrogenated soybean oil, hydrogenated tallow amide, hydrogenated tallow glyceride, hydrogenated vegetable glyceride, hydrogenated vegetable oil, Japan wax, jojoba wax, lanolin alcohol, shea butter, lauramide, methyl dehydroabietate, methyl hydrogenated rosinate, methyl rosinate, methylstyrene/vinyltoluene copolymer, microcrystalline wax, montan acid wax, montan wax, myristyleicosanol, myristyloctadecanol, octadecene/maleic anhyrdine copolymer, octyldodecyl stearoyl stearate, oleamide, oleostearine, ouricury wax, oxidized polyethylene, ozokerite, paraffin, pentaerythrityl hydrogenated rosinate, pentaerythrityl tetraoctanoate, pentaerythrityl rosinate, pentaerythrityl tetraabietate, pentaerythrityl tetrabehenate, pentaerythrityl tetraoleate, pentaerythrityl tetrastearate, ophthalmic anhydride/glycerin/glycidyl decanoate copolymer, ophthalmic/trimellitic/glycols copolymer, polybutene, polybutylene terephthalate, polydipentene, polyethylene, polyisobutene, polyisoprene, polyvinyl butyral, polyvinyl laurate, propylene glycol dicaprylate, propylene glycol dicocoate, propylene glycol diisononanoate, propylene glycol dilaurate, propylene glycol dipelargonate, propylene glycol distearate, propylene glycol diundecanoate, PVP/eiconsene copolymer, PVP/hexadecene copolymer, rice bran wax, stearlkonium bentonite, stearalkonium hectorite, stearamide, stearamide DEA-distearate, stearamide DIBA-stearate, stearamide MEA-stearate, stearone, stearyl erucamide, stearyl stearate, stearyl stearoyl stearate, synthetic beeswax, synthetic wax, trihydroxystearin, triisononanoin, triisostearin, tri-isostearyl trilinoleate, trilaurin, trilinoleic acid, trilinolein, trimyristin, triolein, tripalmitin, tristearin, zinc laurate, zinc myristate, zinc neodecanoate, zinc rosinate, and mixtures thereof. Gelling agents used in vehicles may be natural gelling agents such as natural gums, starches, pectins, agar and gelatin, and may be based on polysaccharides or proteins Examples include but are not limited to guar gum, xanthum gum, alginic acid (E400), sodium alginate (E401), potassium alginate (E402), ammonium alginate (E403), calcium alginate (E404,-polysaccharides from brown algae), agar (E406, a polysaccharide obtained from red seaweeds), carrageenan (E407, a polysaccharide obtained from red seaweeds), locust bean gum (E410, a natural gum from the seeds of the Carob tree), pectin (E440, a polysaccharide obtained from apple or citrus-fruit), and gelatin (E441, made by partial hydrolysis of animal collagen), pentylene glycol 4-t-nutylcyclohexanol (Symsitive 1609).

Pump Spray Composition Example:
0.01 - 5% weight nanoRAD (ACTIVE)
0 - 40% weight ethanol (sub isopropanol - or other drying agent)
0.5 - 2% weight citric acid
0.5 - 3% surfactants
   0.25-2% Lauramine oxide (sub Myristamine oxide or other zwitterionic)
   0-1% tergitol 15-S-15 (non-ionic surfactant: secondary alcohol ethoxylate)
15 - 45% water
1-25% Poly(2-ethyl-2-oxazoline) (Polymer Binder) or similar polymer

In certain embodiments, the nanoRAD spray formulation upon application creates a film that can be rehydrated and shows potential continued disinfecting behavior upon re-hydration. AgCNPs can pull water from gaseous water particles for reactivation, and the polymer film created from the spray formulation is also hydrophilic which assists in achieving a surface water layer from gaseous water particles for reactivation of disinfecting behavior.

According to other embodiments, provided is a container having a pump for dispensing compositions described herein. Pumps may be designed in any manner that meets the limitations of a composition and container, and that dispenses the composition in a desired fashion. Furthermore, pumps may include a tube that extends into the container, thereby facilitating the pumps’ ability to dispense the liquid. Those of skill in the art will appreciate that a pump, including the optional tube, nozzle, and the like, may be in fluid communication with a composition within a container. Pumps may also be designed to be “removably coupled” to a container, meaning that it can be detached and reattached one or more times from the container.

Another embodiment pertains to an apparatus comprising a container portion for holding an amount of a dispensable composition disclosed herein and a nozzle. In a specific embodiment, the apparatus comprises a container suitable for housing a composition; and a pump coupled to the container that includes a nozzle and that is in fluid communication with the composition, the pump being configured to dispense the composition from the nozzle by injecting air into the container to displace the composition. In a specific embodiment, the pump further includes a tube that extends into the container and is in fluid communication with the composition.

In another embodiment, the apparatus comprises a fluid-tight container that is pressurized with a propellant and a valve that dispenses the dispensable composition upon being actuated. The art is well versed in suitable propellants for dispersing compositions. Examples of common propellants include but are not limited to hydrocarbon, ether, compressed gas, chlorofluorocarbon propellant, liquid propellants or mixtures thereof.

Some examples of the types of dispensing containers that may be used in accord with the teachings herein include, but are not limited to, the types of devices disclosed in U.S. Pat. No. 3061202; U.S. Pat. No. 3986644; U.S. Pat. No. 4669664; U.S. Pat. No. 5358179; U.S. Pat. No. 3995778; U.S. Pat No. 4202470; U.S. Pat No. 3992003; CN Pat No. 1042213; U.S. Pat. Pub. 20180370715; U.S. Pat No. 2863699 and U.S. Pat No. 3333743.

### Biocompatibility and Safety

Aware of theconcerns around the toxicology of nanoceria, studies have been conducted regarding the reactivity of cerium salts and this work has spurred an interest in nanotoxicology of cerium oxide [21]. Another study has investigated changes in surface charge and size of CNPs and the impact on cellular uptake.[48] In addition, another study was done using fluorescent conjugates of CNPs that analyzed the kinetics and subcellular localization of nanoceria. [49] Since bare oxide nanomaterials may not be as biocompatible in mammals as soft materials, a study focused on PEG functionalization was done to determine whether PEGylation would alter the catalytic nature of CNPs, and it did not.[50]

There are a variety of methods to synthesize nanoceria particles, including wet chemical, solvothermal, microemulsion, precipitation, hydrolysis and hydrothermal.[51, 52] Based on the synthesis methodology employed, the size of these NPs varies broadly from 3-5 nm to over 100 nm, and the surface charge can vary from -57 mV to +45 mV. The synthesis method can also affect the shape of CNPs. Coatings and surfactants can also be present and contaminate the preparation, such as hexamethylenetetramine (HMT) [53] or ethylene glycol. [54] Many studies that report the toxicity of nanoceria look at NPs generated by hydrothermal methods. This type of CNP typically has sharp edges that can be damaging to cells. [55] However, one of the wet chemical formulations synthesized CNPs that are more biocompatible and observed nearly zero toxicity. This lack of toxicity was observed for human umbilical vein endothelial cells (HUVECs).[56]

While non-toxic neutral pH normo-typic cell, it was still very effective in killing cancer cells [57] due to the acidic chemical environment and nanoceria’s pH-sensitive redox activity. These CNPs also observed the protective effect that had been previously reported for CNPs. In a review article, 38 reports showed protective effects of CNPs in both cell culture and animal studies. [51] It should be noted that many cell types and animal models have been exposed to nanoceria and shown beneficial effects. These include RAW 264.7 macrophages, BEAS-2B lung cells, H9c2 cardiomyocytes, A549 lung cells, HT 22 hippocampal nerve cells, organotypic neurons and many others. Animal models include Tubby mutant mice, EAE model, C57BL/6 mice, Diabetic Wistar rats, and ectopic cancer mouse models.

### EXAMPLES

### Example 1: Formulation of Pure Phase & Silver-Modified Ceria NPs to Induce ROS in Simulated Bio-Fluids

COVID-19 and other flu-like viruses pose a substantial threat to human health due to their high communicability via bio-fluids released from infected individuals. Human to human transfection is especially pronounced in first response and medical environments due to contact with contaminated surfaces in highly trafficked areas. Current disinfectant measures are either unavailable in these environments or show limited efficacy due to mechanistic kinetic limitations. It is shown that nanoceria and Ag-nanoceria will exhibit ROS induction at high reaction rates due to nanoscale/surface effects in presence of virus-laden biofluids. The ROS produced cause a substantial oxidative stress leading to membrane peroxidation and lysis as well as oxidation and inactivation of virus cell receptor surface structures leading to virus inactivation. Literature on nanophase silver and cerium oxide suggest the putative ROS generating reaction scheme under related conditions (Refer to FIG. 1)

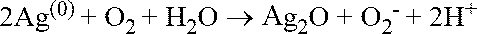

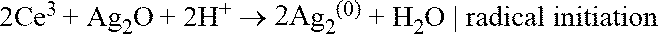

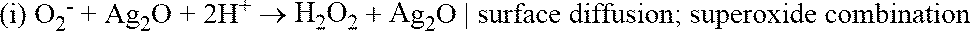

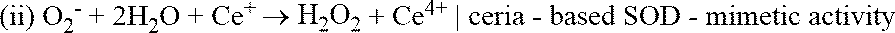

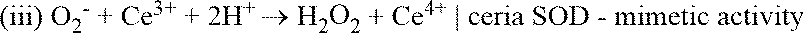

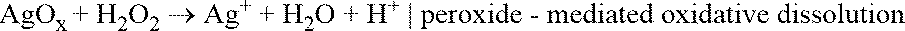

1.1 Synthesis of particles via varied solution-based routes & preliminary characterization is performed. Given the strong influence of chemical environment on nanomaterial surface chemistry and the implications of cerium oxide redox ratio (i.e. relative material composition with respect to Ce³⁺ and Ce⁴⁺ fractions) demonstrated in nanomedicine literature to promote unique ROS generation, several synthesis methods are investigated. Pure phase nanoceria are synthesized via several unique methods previously shown to induce ROS production. In one example, a hydrogen peroxide-based oxidation reaction is used to produce a high Ce³⁺/Ce⁴⁺ ratio nanoceria formulation. In particular, cerium nitrate hexahydrate is dissolved to 5 mM in water followed by addition of 3% hydrogen peroxide under agitation. Particles are left standing over a certain period to allow degradation excess peroxide by the ceria surface.

To produce a more Ce⁴⁺-rich formulation, a second synthesis utilizing a forced hydrolysis approach is performed. Specifically, particles are formed in aqueous solution from a cerium nitrate hexahydrate precursor. Hydrogen peroxide limits the formation of metallic and oxide silver phases (i.e. prevents formation of secondary, distinct silver nano-phases). Therefore, several syntheses will utilize peroxide as an oxidant in silver-modified nanoceria formulations. First, a formulation is produced via an in-situ method wherein cerium and silver nitrates are dissolved followed by direct hydrogen peroxide oxidation and ageing to allow peroxide degradation via cerium oxide surface catalysis. Second, a hybrid forced hydrolysis approach is conducted wherein the dissolved salts are first oxidized via peroxide and subsequently precipitated via addition of 30% ammonium hydroxide. Particles are collected through centrifugation at 10,000 rpm and washed three times with de-ionized water. The combination of direct peroxide-mediated oxidation and a forced hydrolysis approach will mediate changes to cerium redox state ratio. Third, a solution is prepared wherein co-dissolved cerium and silver nitrates undergo ammonium hydroxide-mediated oxidation/precipitation, followed by washing and re-suspension in de-ionized water. From here, hydrogen peroxide is added, and the solution left under stirring to promote the dissolution of secondary phase silver nanomaterials. Particles are subsequently washed to remove ionized silver. Oxidation via peroxide or ammonium hydroxide form oxide particles via unique chemistry and thereby strongly affect the product Ag- nanoceria. The influence of silver fraction (mass percent; 2, 5, 10, 20%) is investigated in each nanomaterial candidate formulation. Particle size and surface charge are evaluated via dynamic light scattering and zeta-potential measurements. Additionally, silver phase character and Ce³⁺/Ce⁴⁺ is evaluated qualitatively via monitoring peaks at ~320 and 252/298 nm, respectively (FIG. 4).

1.2 Formulations generated in 1.1 are assayed for ROS generation chemical activities. In particular, catalase and superoxide dismutase activities are assessed using standard bio-assay kits. Hydroxyl radical generation activity is assessed via assay as degradation of added methylene blue dye. Assays are performed in model bio-fluid solutions (e.g. NaCl/HCl buffer solution at pH 6 and room temperature). Reactions rates related to each reaction are collected and compared. The implications of silver release/ionization in these reactions is assessed. Ionization reactions are monitored first by UV-Vis measurements at regular timepoints (i.e. analyzing silver ion peak evolution) and subsequently via spectro-electrochemistry (i.e. monitoring UV-Vis peak character while simultaneously performing amperometry at open circuit potential and voltammetry/Tafel analysis to detail silver corrosion processes). Additionally, the influence of chloride concentrations on reactions (rates) is assayed via titration and Tafel analysis. Efficacy of the nanomaterial to induce lipid peroxidation is assayed using a commercial Lipid Peroxidation Assay kit (MDA assay). The collective results of these studies are used to modify synthesis parameters from 1.1 to generate Ag- CNP formulations which elicit high reaction rates for ROS production.

### Example 2: Characterize Nanoparticles and Analyze for Efficacy and Toxicity

Preliminary work on CNPs have demonstrated what forms of the CNP lead to different types of biological behavior. It is shown that CNPs and Ag-CNPs will generate ROS which will inactivate the phospholipid bilayer of enveloped viruses - this causes rapid and extensive lysis and inactivation of this class of viruses such that they cannot infect cells.

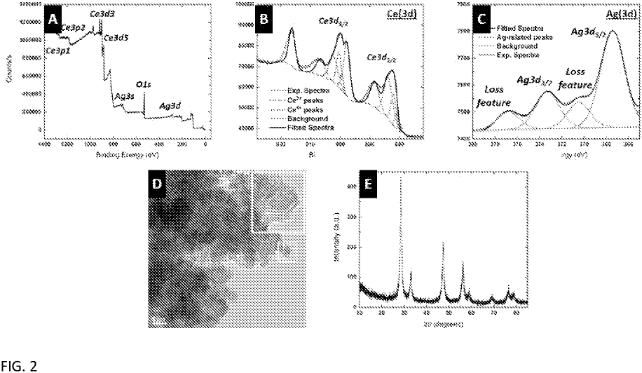

2.1 Formulations demonstrating high rates of ROS producing reactions are characterized with respect to size, morphology, and chemical composition. High-resolution transmission electron microscopy (hrTEM; to demonstrate nanomaterial size, morphology, and grain character), small angle x-ray diffraction (SAXS; to characterize silver and cerium oxide phase crystalline character), and x-ray photoelectron spectroscopy (XPS; to analyze/evaluate chemical composition, cerium redox ratio, and silver oxidation/chemical environment as demonstrated in FIG. 2).

2.2 CNP and Ag-CNP, are evaluated for reducing infectivity by plaque assay or TCID50 assay from solution-suspended virus species. From here, RT-PCR is used to assay viral genomes. Dose- and time-dependence of virus inactivation is established for each formulation.

Two approaches are taken to determine the ability of CNP and Ag-CNP to inactivate a range of human pathogenic viruses. First, various concentrations of virus are incubated in solution with a set concentration of either CNP or Ag-CNP. At various times after mixing, aliquots are removed from the sample, diluted and assayed for remaining infectivity. Whether plaque assays or TCID50 is used depends on the virus. Real time PCR is used to determine the remaining particles irrespective of infectivity. Samples are analyzed in triplicate and data is expressed as fold change in infectivity compared to starting level of virus as shown in our prior publications. [61, 62] Temperature is a major factor in virus stability and is tested along with time of incubation and concentration of CNP or Ag-CNP.

The above assays are carried out first with prototypic lab strains of Coronavirus, so that rapid progress and show productivity can be made. To define the anti-viral specificity of CNP, virus #2 (Zika virus) is tested to determine whether other enveloped positive-sense RNA viruses with structures similar to CoV are also sensitive to inactivation. Virus #3 (rhinovirus) tests whether sensitivity extends to a positive sense RNA virus that lacks a lipid bilayer. Virus #4 (influenza A virus) tests sensitivity of enveloped negative sense RNA viruses, a result which will have implications for mechanism of action. Virus #5 (Vaccinia virus VV) tests inactivation of DNA-containing enveloped viruses. Based on published work [61] (Bracey et al, 2019) showing VV was much more resistant to chemical treatment than RNA viruses, it is anticipate seeing a gradient of sensitivity - CoV>Influenza>VV. The outcome from non-enveloped Rhinovirus will be an important, as this will direct to future studies on whether inactivation is lipid-dependent or nucleic acid-dependent.

If inactivation of any of the enveloped RNA viruses (e.g., coronavirus, Zika virus, influenza virus) is seen, it shows that the envelope has been destroyed by the CNP or Ag-CNP. This involved sucrose gradient sedimentation of samples that include CNP alone, virus alone and CNP plus virus incubated as determined above. After centrifugation, fractions are collected and analyzed by western blotting for the position of the viral components. Intact virus sediments to near the bottom of the tube, whereas disrupted virions remain at the top of the gradient. The direct interactions of CNP with virions is detected by crosslinking experiments and by testing gradient fractions for co-sedimentation of CNP with particles.

### Example 3: Formulation of Optimized Silver-Modified Ceria NPs Aerosol & Support Components

An aerosol or pump spray mediated dispersal of disinfectant agents allows rapid, broad deployment to general surfaces without significant concern for material character or topology. Inclusion of Ag-CNPs into aerosol or spray formulations will function as a portable system for disinfection of general surfaces with high rates of disinfection with continuing residual disinfectant activity upon drying. Further, the storage of such nanomaterials in aerosol media will mediate long shelf-lives for nanomaterial active components; thereby preserving activity prior to administration.

3.1: Ag-CNPs are dispersed in solvents (e.g. alcohols, ethers) of varying volatilities. Depending on the particle preparation method, dispersion is either accomplished by suspending particles in the candidate dispersants following washing steps or through dialysis to remove water phase. Colloidal stability is assessed via dynamic light scattering (i.e. measurements of particle solvo-dynamic radii and aggregation character as change in size relative to hrTEM measurements) and zeta potential (solvent coordination at surface effecting stabilization; zeta potentials > 25 mV considered highly stable). Innocuous ligand species (e.g. non-reactive small, polar organic species such as saccharides) may be added to impart greater stability by coordinating particle surfaces. Optimal dispersant (or propellant) is based on greater volatility (thereby mediating effective hydration by virus-containing bio-fluids following spraying) and nanoparticle colloidal stability.

3.2 Ag-CNPs are suspended in dispersant medium and diluted in bio-fluid model solution. ROS generation is monitored via assay over time to approximate efficacy during vaporization of carrier medium. Rates of reaction are compared relative to activity in pure model medium.

### Example 4: Optimization of Formula for Surfaces and Film Capabilities

Different methods of temporary film forming from formulation solution are possible. These include weak film formation from formulation suspension, van der Waals adhesion of Ag-CNPs to a surface, and weak electrostatic interactions of the NPs to the surface. The small crystallite nature of the active component of the formulation will allow for temporary film formation based on one or more of these mechanisms.

4.1 Spray formulation efficacy on virus-laden surface & dried formulation efficacy as film upon virus/bio-fluid administration. A virus-inoculated test surface is sprayed with the test formulation to determine initial efficacy. The efficacy of the spray as a (dried) film is assayed by dispersing particles on a test surface, followed by inoculation with virus and determination of virus infectivity post-interaction.

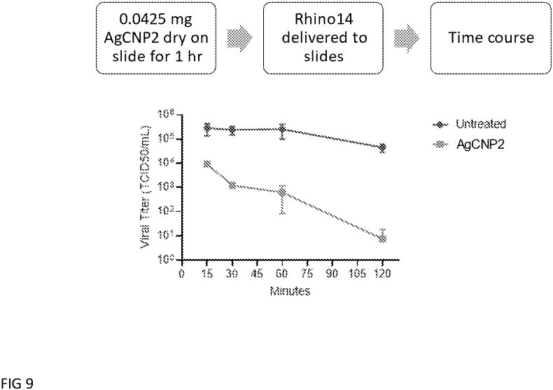

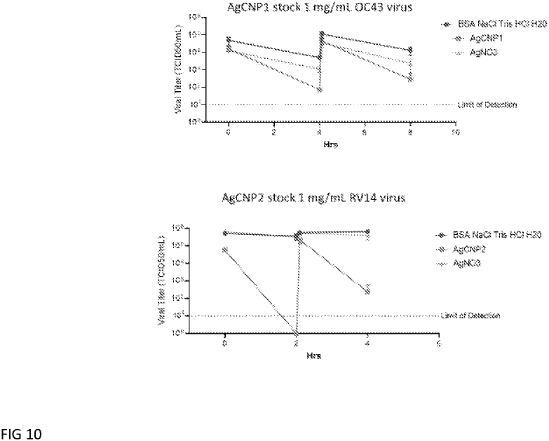

Films are incubated for various time and processed as described above for infectivity and overall particles. AgCNP2 was applied to a slide and allowed to dry for 1 hour. Rhino14 was delivered to the AgCNP2 treated slide and an untreated slide. Over the course of two hours, the viral titer on the AgCNP2 slide decreased at a significantly higher rate than the untreated slide (FIG. 9). A residual efficacy assay of AgCNP1 and 2 against OC43 and RV14 respectfully showed that the AgCNP retain their efficacy over multiple hours (FIG. 10).

### Example 5: Optimization of Metal Mediated Nanoceria Inactivate Human Coronavirus and Rhinovirus by Surface Disruption

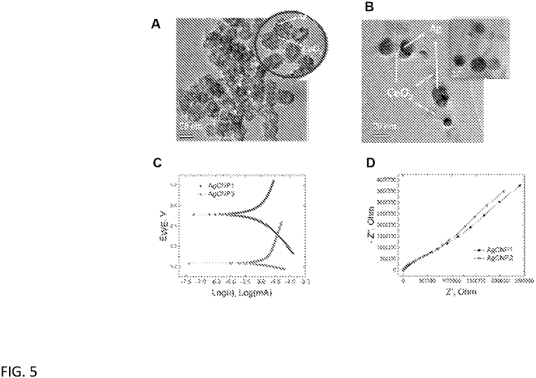

In the presented study, two unique formulations (AgCNP1, AgCNP2) of silver-modified cerium oxide nanoparticles are produced, characterized, and tested for anti-viral efficacy (FIG. 5). Microscopy and photoelectron spectroscopy show clear differences in the redox state composition of cerium, the size of formulation particles, and the presentation of silver phases in ceria matrix. Electrochemical and bandgap measurements provide insight into the nature of silver and silver/ceria interfaces, along with providing evidence of their stabilization by the cerium oxide phase. Anti-viral efficacy was determined across a set of unique virus types with the AgCNP formulations showing specificity towards particular viruses in their anti-viral activities. Herein, anti-viral efficacies against rhinovirus RV14 and coronavirus OC43 are determined and compared. For the first time, in situ electrochemical impedance spectroscopy methods were performed and corroborate the specificity of AgCNP formulation/virus type interactions over incubation periods. From this data, along with results from a designed analogous system, general modes of mechanism action are determined for describing anti-viral activities for both high-efficacy virus/AgCNP formulation pairs.

### 5.1 Materials Synthesis & Colloidal Character

Silver modified cerium oxide formulations (AgCNPs) were synthesized in two unique formulations (AgCNP1, AgCNP2) each utilizing different chemical reactions specific to aqueous silver. AgCNP1 was synthesized via a previously developed, two step procedure (FIG. 5). Briefly, a solution containing AgCNP-like, silver-modified nanoceria, and silver secondary phases are formed via an alkaline-forced hydrolysis reaction. The product materials are washed with dH₂O and subsequently treated with ammonium hydroxide. Ammonium hydroxide functions as an etchant as well as a phase transfer complex: mediating the solubilization/stabilization of dissolved silver ions in the aqueous phase. In particular, the reaction results in the formation of Tollen’s reagent (Ag[(NH₃)₂OH]_{aq}). The resulting single particle solution is then washed with dH₂O to remove excess base and counter/spectator ions. AgCNP2 utilizes the stability of silver ions towards oxidation by hydrogen peroxide. Specifically, dissolution of cerium and silver nitrates followed by addition of hydrogen peroxide leads to the selective oxidation of cerium ions over silver and the evolution of metallic silver phases on the ceria surface. The unique synthesis conditions of these particles suggest a potentially disparate particle character.

Colloidal characters of the particles were evaluated for kinetic stability, surface potential, and hydrodynamic diameter. Dynamic light scattering (DLS) measurements of each sample are collected in Table 3 and relate a greater particle size (including specific spheres of hydration) for each formulation with AgCNP1 particles being ~3x larger in diameter (Table 3). Further, zeta potential measurements indicate a greater surface potential for AgCNP2 over AgCNP1, each with a positive polarity. These characterizations suggest the observation that AgCNP2 particles show greater kinetic stability over AgCNP1 (AgCNP1: moderate precipitation following particle ageing 1 week in room condition; AgCNP2: no observable sedimentation for greater than 5 months). AgCNP1 particles also present as turbid in solution at 1 mg/mL whereas AgCNP2 are completely translucent under similar conditions, suggesting greater Mie scattering related to larger particle size. Particles from each synthesis were observed to demonstrate unique fundamental and functional material character.

TABLE 3

Physicochemical Properties of AgCNP formulations

| | | |
|---|---|---|
| | AgCNP1 | AgCNP2 |
| Ce3+: Ce4+ (%Ce³⁺) | 25.8% | 53.7% |
| [Ag]/[Ag+Ce] | 16.9% | 14.6% |
| SOD Activity (% Inhibition) | 97.9% | 99.2% |
| (Hydrodynamic Diam. (nm)) | 42.2±4.6 | 16±5.1 |
| Zeta Potential (mV) | 22.4 ± 0.9 | 24.1 ± 1.3 |
| Ecorr | 465.386 mV | 217.374 mV |
| Icorr | 0.027 uA | 0.013 uA |
| Betaₐ | 681.7 mV | 617.0 mV |
| Beta_{c} | 269.2 mV | 21.8 mV |

### 5.3 Electrochemical Characterization

XPS results suggest a unique silver character for each formulation and therefor, the stability of silver phases for each formulations were evaluated via common electrochemical techniques. Electrochemical measurements (FIGS. 5C, D) were performed to determine the activity of silver phases in AgCNP formulations along with their susceptibility to electron transfer processes. In corroboration with XPS results, AgCNP1 evidenced a larger Tafel potential (Table 3) than AgCNP2 (465.4 vs. 217.4 mV, respectively) suggesting a greater stability towards electron transfer and a more noble oxidation character. Interestingly, AgCNP1 demonstrated a Tafel current (which was twice the value observed for AgCNP2 (0.027 and 0.013 µA, respectively). These values are relatively low suggesting an overall stability for silver phases in each formulation. However, the greater current value for AgCNP1 at higher potential may be understood from XPS spectra wherein a fraction of sample silver content was found as an oxide. Penetration of silver into the ceria surface/sub-surface would increase the Tafel potential (i.e. have a stabilizing effect on the silver phase) while simultaneously improving registry of the phases at their interphase, improving charge transfer as Tafel current. TEM images confirm the greater interfacial area for silver-ceria in AgCNP1 over AgCNP2. Significantly greater anodic β values over cathodic were observed in Tafel analysis (Table 3) for both samples suggesting electron transfer at Tafel potentials are kinetically favored by oxidation processes. While electrochemical methods can provide information on fundamental charge transfer processes, this characterization provides only nominal information at the atomistic or chemical level.

### 5.5 Selective Inactivation of Two Human Respiratory Viruses With AgCNP1 and AgCNP2

To determine the extent to which nanoceria and silver-modified nanoceria can inactivate human coronavirus OC43, reactions were prepared to include 10⁵ infectious units (TCID50) of virus per ml, together with buffer and nanoparticles. Alternatively, buffer alone reactions were included with water as a vehicle control. The 10⁵ TCID50 /ml input virus was determined as time zero infectivity. After 6 hr incubation, the buffer alone control reactions had 10⁴TCID50 /ml remaining infectious virus. The unmodified nanoceria, CNP2 and CNP1, had little effect on virus titer with reactions remaining at about 5*10⁴TCID50 /mL. Strikingly, AgCNP1 treatment resulted in complete inactivation of infectious virus, whereas AgCNP2 treatment reduced infectious virus titer to ~10³ TCID50 /mL. A time course study was conducted with reactions prepared as described above to include buffer alone, AgCNP1, or AgCNP2. Infectious virus was determined after incubation for zero, 2, 4, and 6 hr. As early as 4 hr, AgCNP1 treatment reduced OC43 virus titer from an initial value of 10⁵ TCID50 /mL to less than 10² TCID50 /mL. Taken together, these data suggest AgCNP1 was highly effective at inactivating coronavirus OC43 and that AgCNP2 had a modest capacity for inactivation of OC43.

To determine the optimal effective AgCNP1 concentration to inactivate coronavirus OC43, reactions were prepared starting with 10⁵ TCID50 /mL of OC43, along with buffer and increasing concentrations of AgCNP1. Infectious virus was determined after 5 minute and 4 hr incubations. All AgCNP1 concentrations had similar virus titers around 10⁴-10⁵ TCID50 /mL after the 5 minute time point. By contrast, a 4 hr treatment with 0.77 mg/mL AgCNP1 resulted in no detectable OC43 virus infectivity and 0.2 mg/mL AgCNP1 treatment reduced infectivity to ~10²TCID50 /mL. Results of AgCNP1 -OC43 inactivation were confirmed using an alternative measure of infectivity. A 4 hr incubation of 10⁴ Plaque Forming Units (PFU)/mL of OC43 with buffer alone recovered all infectivity, compared to incubation with 0.77 mg/mL AgCNP1 which resulted in no detectable OC43 plaques in the assay. Taken together, these data show both time- and dose-dependent inactivation of coronavirus OC43 infectivity by AgCNP1.

We next sought to determine the extent to which nanoceria and silver-modified nanoceria can inactivate the human respiratory pathogen rhinovirus 14 (RV14), a non-enveloped icosahedral RNA virus. RV14 was incubated with buffer alone or with nanoparticles shown. Buffer alone reactions were prepared with water as a vehicle control. 6*10⁵ TCID50 /mL input RV14 virus was determined and represented as time zero. After 6 hr incubation, the buffer alone reactions retained the input infectivity of 6*10⁵ TCID50 /mL. The unmodified nanoceria, CNP2 and CNP1, had little effect on RV14 infectivity. Importantly, AgCNP1 treatment reduced infectious virus titer to 5*10² TCID50 /mL, whereas AgCNP2 treatment resulted in complete inactivation of infectious virus. In a time course study, incubation of 6*10⁵ TCID50 /mL of RV14 with buffer alone showed no loss of infectivity over a 6 hr incubation. By contrast, there was a very rapid loss of RV14 infectivity to undetectable levels by 2 hr incubation with AgCNP2. Incubation with AgCNP1 showed slower inactivation of RV14 compared to AgCNP2, with virus titer being reduced to -10² TCID50 /mL after 6 hrs. Taken together, these data demonstrate that both AgCNP1 and AgCNP2 can inactivate RV14 infectivity, with AgCNP2 having a more potent anti-RV14 effect.

### 5.6 In Situ Bio-Electrochemical Impedance Spectroscopy Characterization of AgCNP Disinfectant Activity

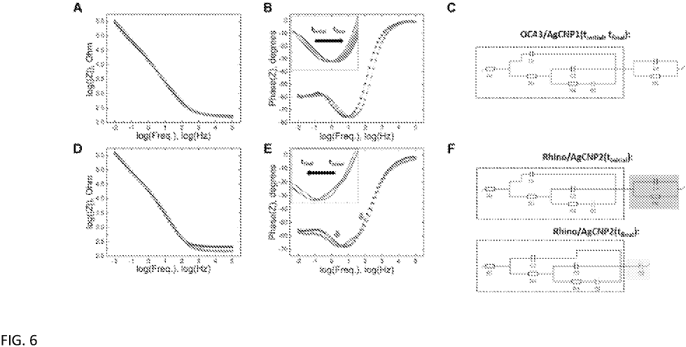

The substantial disinfectant activities demonstrated by both formulations to unique subsets of virus species, along with HA assay results, suggest unique modes of action in each test case. In order to probe the character of each, electrochemical impedance spectroscopy (EIS) was performed for two test cases; namely, AgCNP1/OC43 and AgCNP2/Rhinovirus (FIG. 6). EIS is a non-destructive characterization technique that relies on the application of a small amplitude potential at frequencies varied over a fixed range. Decomposition of measured currents into contributions from unique frequency regions allows the determination of characteristic electrochemical processes. EIS is a staple technique in the manufacturing sector and in particular for the energy and semiconductor industries. Herein, total impedance is measured with the data fit to simple circuit diagrams (i.e. with fit circuit elements representing chemical components/processes). In recent years, the technique has been applied to the study of changing cell character upon physical or chemical stimulation. Among these studies, conditions wherein the cell membrane character changes are the most often investigated. A simple interpretation of cell-substrate EIS data is given by the ECIS model of Giaever and Keese wherein impedance components are de-convoluted as resistance to charge flow between biological particles, as well as from regions between particles and the electrode substrate, and cell membrane capacitance. In studies of cell health, these model components are diagnostic: with each changing upon introduction of toxic agents (e.g. membrane pore-formation, retraction of focal regions, membrane oxidation). Further, these responses are necessarily frequency dependent with specific frequency bands identified for unique biological processes. Three regions in particular are highlighted and represented as α (< 10 kHz), β (10 kHz < 100 mHz), and γ (GHz). In identifying changes to impedance spectra over time in presence of test agents (e.g. AgCNPs), specific biochemical processes may be identified.

In the presented study, test case impedance spectra were unique from each other (FIG. 6). For AgCNP1 (FIGS. 6A-B), spectra collected over the 8 hr disinfection period used for infectivity assays, described above. The spectra show a near-consistent impedance character with differences in magnitude only being evident at high frequencies (decreasing with time; 100 Hz to 100 kHz), in Bode representation. In phase versus log(frequency) representation, there is a clear, time-dependent shift in phase peak to higher frequency. These results being limited to the α-dispersion region: we expect spectra changes to be associated with ionic diffusion, especially in the cell membrane, as well as physical interactions with the cell membrane. The peak-like spectrum feature represents a superposition of two physical processes with different time-constants which can be ascribed to specific changes at the cell membrane through fitting and circuit modeling (below). AgCNP2 (FIGS. 6D-E) shows a similar initial spectrum character (two component) over the 4 hr incubation period. However, with increasing incubation time the spectrum becomes more complex: appearing as two observable “peaks” which can be resolved into a four-component function. Differences between these spectra corroborate the disparate particle-virus interactions and suggest the presence of an additional physical element. Given the observed phase shift to higher frequencies, data suggests a constant phase element component (impedance being dominated by resistance at increasing frequencies). Fittings of the spectra demonstrate these characters with a common diagram construction for all test cases save for the variable elements (shown enclosed by dotted lines in FIGS. 6C,F) which are particular to specific AgCNP and virus interactions at unique time points (FIGS. 6C,F). The variable elements being fit as a parallel resistor and capacitor for AgCNP1:OC43 and as a constant phase element for AgCNP2:RV14, as suggested by the phase v. impedance character of the spectra. In particular, the parallel elements fit to the time-dependent behavior of the AgCNP1:OC43 interaction change in value from high resistance and moderate capacitance to significantly lower values of each. In particular, the resistance value changes precipitously with incubation. The results together corroborate the proposed particle: virus interaction leading to changes in membrane integrity/permeability; decreasing resistance being related to the permeability and capacitance to the lowered membrane density and physical interaction with the oxide nanoparticle. The constant phase element variable component of RV14:AgCNP2 is a frequency dependent element that models an imperfect dielectric. In the case of this system, increasing incubation team leads to an increasingly imperfect character for the model dielectric: leading to evolution of a resistive character from the initial character similar to that seen for OC43. In order to better interpret and assign the observed in situ character to unique physicochemical process, a physical model was produced and unique control reactions studied.

### 5.7 Developing a Physical Model of Bio-Electrochemical Impedance Spectroscopy

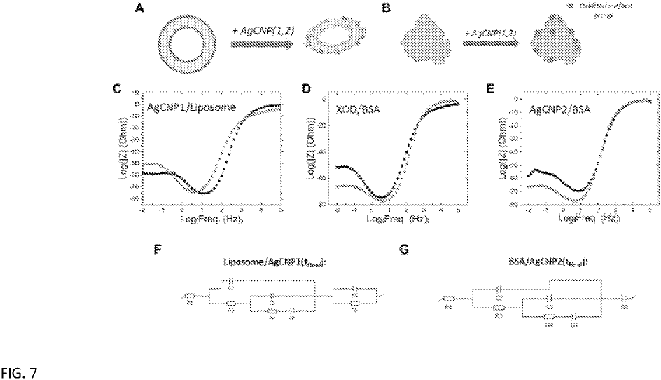

Analog systems were produced with respect to RV14 and OC43 virus systems to identify the unique anti-viral mechanisms produced during in situ EIS measurements. Specifically, we looked to reproduce the character of the viruses at the interface between the virus and the electrolyte. Therefore, two unique systems were produced to model the dense protein structure of the RV14 surface and the enveloped surface of OC43. For measurements related to RV14, bovine serum albumin was used while liposomes were used for the lipid membrane of OC43. All measurements were performed in identical electrolyte conditions as those for the in situ measurements to control for solution-based impedance contributions (i.e. 0.1 M Tris-HCl, pH 7.5). Liposomes are commonly used in virus studies, including as virus-mimetic vectors for drug/gene delivery therapies, as virus-like particles. In the current study, liposomes were synthesized to the approximate dimensions (~120 nm) of the OC43 coronavirus to appropriately model any physical interaction between the AgCNP and the liposome. In each test case, the virus-analog material was dispersed in solution and dropcast to the surface of a glassy carbon electrode in a manner similar to the protocol used for the in situ virus measurements. In each case the behavior of the analog material seemed to reflect the behavior observed for the related virus, with the corresponding AgCNP formulation dependance. FIG. 4 shows the collected EIS spectra for the virus analog measurements of virus:particle pairs which were effective in infectivity assays. It is notable that the fitted spectra lead to equivalent circuits similar to the in situ data. In particular, the circuit diagrams are identical with those produced in the in situ study, with only the elements at right of the diagram remaining variable. For the Liposome/AgCNP1 system (FIGS. 7A,F) we observe the variable elements are a parallel resistor and capacitor with this character retained over the incubation period. However, we see the values of these elements change over the incubation period resulting in a related phase shift due to change in character from more capacitive to resistive. Related fitted materials for the BSA/AgCNP2 system (FIGS. 7E,G) occur and relate to the RV14/CNP2 in situ data. However, we see that the spectra in the analog system is less defined than that seen in the virus system. The slight variation in character can be ascribed to the small-scale (topological) differences among the system. In particular, BSA is a single globular protein while RV14 is an aggregate of proteins, with a rougher surface topology. Differences among the spectra may be ascribed to the varied physicochemical environment, however, spectra suggest that the total interaction between the particle and virus/analog are similar. Given the evolution of additional resistive character in the models, we determined to identify any specific chemical changes occurring. Therefore, an oxygen radical generating system, known to induce lipid peroxidation through the simultaneous proportional production of superoxide and hydrogen peroxide was used as a positive control for the activity.

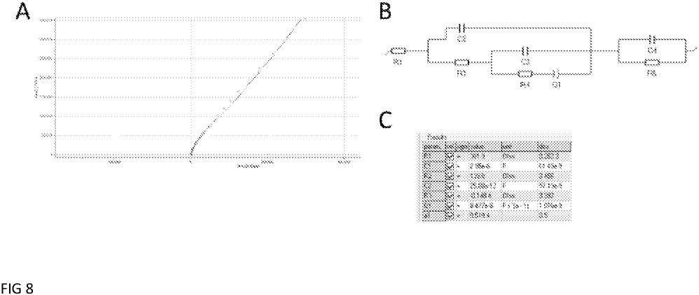

In these experiments, the effects of the positive control for radical oxygen evolution were assessed via related changes in the spectrum. It was observed (FIG. 7B) that oxidation reproduced the observed additional peak observed in the RV14/AgCNP2 system for the BSA/AgCNP2 spectrum (FIG. 7C). The observed character was also reproduced for the Lipo/AgCNP1 system (FIG. 8), confirming that the spectra character change in the viral system does not originate from a chemical attack in AgCNP1 incubation.

### REFERENCES

https://www.cdc.gov/coronavirus/2019-ncov/community/organzations/cleaning-disinfection.html.

https://www.nih.gov/news-events/news-releases/new-coronavirus-stable-hours-surfaces.

https://www.epa.gov/pes ticide-registration/list-n-disinfectants-use-against-sars-cov-2.

https://timesofindia.indiatimes.com/city/kolkata/new-market-sprinklers-to-spray-disinfectant-solution-on-shop pers/articleshow/75017812.cms.

Antiseptics and Disinfectants Market Size, Share & Trends Analysis Report By Type (Quaternary Ammonium, Chlorine), Product Type (Medical Device Disinfectants), By End Use, And Segment Forecasts, 2019 - 2026.

https://nrckids.org/files/appendix/AppendixK.pdf.

https://www.cdc.gov/coronavirus/2019-ncov/community/schools-childcare/guidance-for-childcare.html#CleanDisinfect.

R.A. Yokel, S. Hussain, S. Garantziotis, P. Demokritou, V. Castranova, F.R. Cassee, The yin: an adverse health perspective of nanoceria: uptake, distribution, accumulation, and mechanisms of its toxicity, Environmental Science: Nano 1 (5) (2014) 406-428.

W.T. Self, E. Bossy-Wetzel, S. Seal, J. Dowding, Neuronal protection by cerium oxide nanoparticles, Google Patents, 2018.

W.T. Self, S. Seal, Nanoparticles of cerium oxide having superoxide dismutase activity, Google Patents, 2009.

B.A. Rzigalinski, S. Seal, D. Bailey, S. Patil, Cerium oxide nanoparticles and use in enhancing cell survivability, Google Patents, 2009.

S. Seal, H. Cho, S. Patil, A. Mehta, Cerium oxide nanoparticle regenerative free radical sensor, Google Patents, 2012.

P. Brenneisen, S. Seal, A. Karakoti, Redox active cerium oxide nanoparticles and associated methods, Google Patents, 2017.

Cerium Oxide Nanoparticles Market: Market by Application (Energy Storage, Polishing Agent, Personal Care, Pharmaceuticals); Report ID: GVR-1-68038-547-2, August 2017.

D.-s. Tsai, T.-S. Yang, Y.-S. Huang, P.-W. Peng, K.-L. Ou, Disinfection effects of undoped and silver-doped ceria powders of nanometer crystallite size, International journal of nanomedicine 11 (2016) 2531.

E. Grulke, K. Reed, M. Beck, X. Huang, A. Cormack, S. Seal, Nanoceria: factors affecting its pro-and anti-oxidant properties, Environmental Science: Nano 1 (5) (2014) 429-444.

G. Sener, S.A. Hilton, M.J. Osmond, C. Zgheib, J.P. Newsom, L. Dewberry, S. Singh, T.S. Sakthivel, S. Seal, K.W. Liechty, Injectable, self-healable zwitterionic cryogels with sustained microRNA-cerium oxide nanoparticle release promote accelerated wound healing, Acta biomaterialia 101 (2020) 262-272.

V. Shah, S. Shah, H. Shah, F.J. Rispoli, K.T. McDonnell, S. Workeneh, A. Karakoti, A. Kumar, S. Seal, Antibacterial activity of polymer coated cerium oxide nanoparticles, PLoS One 7(10) (2012).

L. Alili, M. Sack, A.S. Karakoti, S. Teuber, K. Puschmann, S.M. Hirst, C.M. Reilly, K. Zanger, W. Stahl, S. Das, Combined cytotoxic and anti-invasive properties of redox-active nanoparticles in tumor-stroma interactions, Biomaterials 32(11) (2011) 2918-2929.

C. Korsvik, S. Patil, S. Seal, W.T. Self, Superoxide dismutase mimetic properties exhibited by vacancy engineered ceria nanoparticles, Chemical Communications (10) (2007) 1056-1058.

E.G. Heckert, A.S. Karakoti, S. Seal, W.T. Self, The role of cerium redox state in the SOD mimetic activity of nanoceria, Biomaterials 29(18) (2008) 2705-9.

J. Chen, S. Patil, S. Seal, J.F. McGinnis, Rare earth nanoparticles prevent retinal degeneration induced by intracellular peroxides, Nat Nanotechnol 1(2) (2006) 142-50.

T. Pirmohamed, J.M. Dowding, S. Singh, B. Wasserman, E. Heckert, A.S. Karakoti, J.E.S. King, S. Seal, W.T. Self, Nanoceria exhibit redox state-dependent catalase mimetic activity, Chemical Communications 46(16) (2011) 2736-2738.

S. Babu, J. Cho, J. Dowding, E. Heckert, C. Komanski, S. Das, J. Colon, C. Baker, M. Bass, W. Self, S. Seal, Multicolored redox active upconverter cerium oxide nanoparticle for bio-imaging and therapeutics, Chemical Communications 46(37) (2010) 6915-6917.

S. Seal, Lanthanide doped nanocrystalline ceria coating for increasing oxidation resistance of stainless steel and associated methods, Google Patents, 2013.

M.H. Kuchma, C.B. Komanski, J. Colon, A. Teblum, A.E. Masunov, B. Alvarado, S. Babu, S. Seal, J. Summy, C.H. Baker, Phosphate ester hydrolysis of biologically relevant molecules by cerium oxide nanoparticles, Nanomedicine: Nanotechnology, Biology and Medicine 6(6) (2010) 738-744.

A. Karakoti, S. Singh, J.M. Dowding, S. Seal, W.T. Self, Redox-active radical scavenging nanomaterials, Chemical Society Reviews 39(11) (2010) 4422-4432.

Q. Fu, H. Saltsburg, M. Flytzani-Stephanopoulos, Active nonmetallic Au and Pt species on ceria-based water-gas shift catalysts, Science 301 (5635) (2003) 935-938.

S. Seal, E.L. Petersen, S. Deshpande, S. Patil, S.C. Kuiry, Use of oxide nanoparticles in soot reduction, Google Patents, 2008.

C.T. Campbell, C.H.F. Peden, Chemistry - Oxygen vacancies and catalysis on ceria surfaces, Science 309(5735) (2005) 713-714.

F. Esch, S. Fabris, L. Zhou, T. Montini, C. Africh, P. Fornasiero, G. Comelli, R. Rosei, Electron localization determines defect formation on ceria substrates, Science 309(5735) (2005) 752-755.

A. Kumar, S. Babu, A.S. Karakoti, A. Schulte, S. Seal, Luminescence properties of europium-doped cerium oxide nanoparticles: role of vacancy and oxidation states, Langmuir 25(18) (2009) 10998-11007.

U.M. Bhatta, I.M. Ross, T.X. Sayle, D.C. Sayle, S.C. Parker, D. Reid, S. Seal, A. Kumar, G.n. Möbus, Cationic surface reconstructions on cerium oxide nanocrystals: an aberration-corrected HRTEM study, ACS nano 6(1) (2012) 421-430.

T. Sakthivel, S. Das, A. Kumar, D.L. Reid, A. Gupta, D.C. Sayle, S. Seal, Morphological phase diagram of biocatalytically active ceria nanostructures as a function of processing variables and their properties, ChemPlusChem 78(12) (2013) 1446-1455.

S. Deshpande, S. Patil, S.V. Kuchibhatla, S. Seal, Size dependency variation in lattice parameter and valency states in nanocrystalline cerium oxide, Applied Physics Letters 87(13) (2005) 133113.

C. Walkey, S. Das, S. Seal, J. Erlichman, K. Heckman, L. Ghibelli, E. Traversa, J.F. McGinnis, W.T. Self, Catalytic properties and biomedical applications of cerium oxide nanoparticles, Environmental Science: Nano 2(1) (2015) 33-53.

T. Pirmohamed, J.M. Dowding, S. Singh, B. Wasserman, E. Heckert, A.S. Karakoti, J.E. King, S. Seal, W.T. Self, Nanoceria exhibit redox state-dependent catalase mimetic activity, Chemical communications 46(16) (2010) 2736-2738.

M. Hijaz, S. Das, I. Mert, A. Gupta, Z. Al-Wahab, C. Tebbe, S. Dar, J. Chhina, S. Giri, A. Munkarah, Folic acid tagged nanoceria as a novel therapeutic agent in ovarian cancer, BMC cancer 16(1) (2016) 220.

I. Kalashnikova, J. Mazar, C.J. Neal, A.L. Rosado, S. Das, T.J. Westmoreland, S. Seal, Nanoparticle delivery of curcumin induces cellular hypoxia and ROS-mediated apoptosis via modulation of Bcl-2/Bax in human neuroblastoma, Nanoscale 9(29) (2017) 10375-10387.

A. Shcherbakov, N. Zholobak, N.Y. Spivak, V. Ivanov, Advances and prospects of using nanocrystalline ceria in cancer theranostics, Russian Journal of Inorganic Chemistry 59(13) (2014) 1556-1575.

N.M. Zholobak, V.K. Ivanov, A.B. Shcherbakov, Interaction of nanoceria with microorganisms, Nanobiomaterials in Antimicrobial Therapy, Elsevier2016, pp. 419-450.

V. Lozovski, V. Lysenko, V. Piatnytsia, O. Scherbakov, N. Zholobak, M. Spivak, Physical point of view for antiviral effect caused by the interaction between the viruses and nanoparticles, Journal of Bionanoscience 6(2) (2012) 109-112.

A. Arumugam, C. Karthikeyan, A.S. Haja Hameed, K. Gopinath, S. Gowri, V. Karthika, Synthesis of cerium oxide nanoparticles using Gloriosa superba L. leaf extract and their structural, optical and antibacterial properties, Materials Science and Engineering: C 49 (2015) 408-415.

A. Thill, O. Zeyons, O. Spalla, F. Chauvat, J. Rose, M. Auffan, A.M. Flank, Cytotoxicity of CeO2 nanoparticles for Escherichia coli. Physico-chemical insight of the cytotoxicity mechanism, Environmental science & technology 40(19) (2006) 6151-6156.

V. Lozovski, V. Lysenko, V. Pyatnitsia, M. Spivak, Can nanoparticles be useful for antiviral therapy?, Semiconductor Physics Quantum Electronics & Optoelectronics (2011 ).

P. Janoš, J. Ederer, M. Došek, J. Stojdl, J. Henych, J. Tolasz, M. Kormunda, K. Mazanec, Can cerium oxide serve as a phosphodiesterase-mimetic nanozyme?, Environmental Science: Nano 6(12) (2019) 3684-3698.

S. Singh, T. Dosani, A.S. Karakoti, A. Kumar, S. Seal, W.T. Self, A phosphate-dependent shift in redox state of cerium oxide nanoparticles and its effects on catalytic properties, Biomaterials 32(28) (2011) 6745-6753.

A. Vincent, S. Babu, E. Heckert, J. Dowding, S.M. Hirst, T.M. Inerbaev, W.T. Self, C.M. Reilly, A.m.E. Masunov, T.S. Rahman, S. Seal, Protonated Nanoparticle Surface Governing Ligand Tethering and Cellular Targeting, ACS Nano 3(5) (2009) 1203-1211.

S. Singh, A. Kumar, A. Karakoti, S. Seal, W. Self, Unveiling the mechanism of uptake and sub-cellular distribution of cerium oxide nanoparticles, Molecular Biosystems 6(10) (2010) 1813-1820.

A.S. Karakoti, S. Singh, A. Kumar, M. Malinska, S.V. Kuchibhatla, K. Wozniak, W.T. Self, S. Seal, PEGylated nanoceria as radical scavenger with tunable redox chemistry, Journal of the American Chemical Society 131(40) (2009) 14144-14145.

S. Das, J.M. Dowding, K.E. Klump, J.F. McGinnis, W. Self, S. Seal, Cerium oxide nanoparticles: applications and prospects in nanomedicine, Nanomedicine 8(9) (2013) 1483-1508.

C. Sun, H. Li, L. Chen, Nanostructured ceria-based materials: synthesis, properties, and applications, Energy & Environmental Science 5(9) (2012) 8475-8505.

J.M. Dowding, S. Das, A. Kumar, T. Dosani, R. McCormack, A. Gupta, T.X. Sayle, D.C. Sayle, L. von Kalm, S. Seal, Cellular interaction and toxicity depend on physicochemical properties and surface modification of redox-active nanomaterials, ACS nano 7(6) (2013) 4855-4868.

A.S. Karakoti, S. Das, S. Thevuthasan, S. Seal, PEGylated inorganic nanoparticles, Angewandte Chemie International Edition 50(9) (2011) 1980-1994.

E.-J. Park, J. Choi, Y.-K. Park, K. Park, Oxidative stress induced by cerium oxide nanoparticles in cultured BEAS-2B cells, Toxicology 245(1) (2008) 90-100.

S. Das, S. Singh, J.M. Dowding, S. Oommen, A. Kumar, T.X.T. Sayle, S. Saraf, C.R. Patra, N.E. Vlahakis, D.C. Sayle, W.T. Self, S. Seal, The induction of angiogenesis by cerium oxide nanoparticles through the modulation of oxygen in intracellular environments, Biomaterials 33(31) (2012) 7746-7755.

M. Sack, L. Alili, E. Karaman, S. Das, A. Gupta, S. Seal, P. Brenneisen, Combination of conventional chemotherapeutics with redox-active cerium oxide nanoparticles-A novel aspect in cancer therapy, Molecular cancer therapeutics 13(7) (2014) 1740-1749.

J.B. Johnson, D.S. Lyles, M.A. Alexander-Miller, G.D. Parks, Virion-associated complement regulator CD55 is more potent than CD46 in mediating resistance of mumps virus and vesicular stomatitis virus to neutralization, Journal of virology 86(18) (2012) 9929-9940.

C.R. Fox, G.D. Parks, Parainfluenza virus infection sensitizes cancer cells to DNA-damaging agents: implications for oncolytic virus therapy, Journal of virology 92(7) (2018) e01948-17.

J. Mazar, Y. Li, A. Rosado, P. Phelan, K. Kedarinath, G.D. Parks, K.A. Alexander, T.J. Westmoreland, Zika virus as an oncolytic treatment of human neuroblastoma cells requires CD24, PloS one 13(7) (2018).

D.N. Bracey, T.M. Seyler, A.H. Jinnah, T.L. Smith, D.A. Ornelles, R. Deora, G.D. Parks, M.E. Van Dyke, P.W. Whitlock, A porcine xenograft-derived bone scaffold is a biocompatible bone graft substitute: An assessment of cytocompatibility and the alpha-Gal epitope, Xenotransplantation 26(5) (2019) e12534.

P.W. Whitlock, T.M. Seyler, G.D. Parks, D.A. Ornelles, T.L. Smith, M.E. Van Dyke, G.G. Poehling, A novel process for optimizing musculoskeletal allograft tissue to improve safety, ultrastructural properties, and cell infiltration, JBJS 94(16) (2012) 1458-1467.

Walls, A. C.; Park, Y.-J.; Tortorici, M. A.; Wall, A.; McGuire, A. T.; Veesler, D., Structure, function, and antigenicity of the SARS-CoV-2 spike glycoprotein. Cell 2020, 181 (2), 281-292. e6.

Walls, A. C.; Xiong, X.; Park, Y.-J.; Tortorici, M. A.; Snijder, J.; Quispe, J.; Cameroni, E.; Gopal, R.; Dai, M.; Lanzavecchia, A., Unexpected receptor functional mimicry elucidates activation of coronavirus fusion. Cell 2019, 176 (5), 1026-1039. e15.

Baker, A. N.; Richards, S.-J.; Guy, C. S.; Congdon, T. R.; Hasan, M.; Zwetsloot, A. J.; Gallo, A.; Lewandowski, J. R.; Stansfeld, P. J.; Straube, A., The SARS-COV-2 spike protein binds sialic acids and enables rapid detection in a lateral flow point of care diagnostic device. ACS central science 2020, 6 (11), 2046-2052.

Waman, V. P.; Sen, N.; Varadi, M.; Daina, A.; Wodak, S. J.; Zoete, V.; Velankar, S.; Orengo, C., The impact of structural bioinformatics tools and resources on SARS-CoV-2 research and therapeutic strategies. Briefings in bioinformatics 2021, 22 (2), 742-768.

Li, X.; Yu, J.; Zhang, Z.; Ren, J.; Peluffo, A. E.; Zhang, W.; Zhao, Y.; Wu, J.; Yan, K.; Cohen, D., Network bioinformatics analysis provides insight into drug repurposing for COVID-19. Medicine in Drug Discovery 2021, 100090.

Bauer, D. C.; Tay, A. P.; Wilson, L. O.; Reti, D.; Hosking, C.; McAuley, A. J.; Pharo, E.; Todd, S.; Stevens, V.; Neave, M. J., Supporting pandemic response using genomics and bioinformatics: A case study on the emergent SARS-CoV-2 outbreak. Transboundaryand emerging diseases 2020, 67(4), 1453-1462.

Robson, B., Bioinformatics studies on a function of the SARS-CoV-2 spike glycoprotein as the binding of host sialic acid glycans. Computers in biology and medicine 2020, 122, 103849.

1. A dispensable composition comprising metal-associated cerium oxide nanoparticles (mCNPs) and an excipient wherein m is a metal and a fluid of the excipient and the mCNPs have ionized metal removed.

2. The dispensable composition of claim 1, wherein the metal is selected from the group consisting of silver, gold, ruthenium, vanadanium, copper, titanium, nickel, platinum, titanium, tin and iron.

3. The dispensable composition of claim 2, wherein the metal comprises a stable metallic silver (AgCNPs).

4. The dispensable composition of claim 3, wherein the silver is in an amount of less than 10% by weight.

5. The dispensable composition of claim 1, wherein the excipient is selected from the group consisting of water, chloroform, methylene chloride, acetone, methyl ethyl ketone, cyclohexane, ethyl acetate, diethyl ether, lower alcohols, lower diols, THF, DMSO, or DMF.

6. The dispensable composition of claim 5, wherein the excipient comprises water.

7. The dispensable composition of claim 1, wherein the mCNPs are further doped with fluorine.

8. The dispensable composition of claim 1, wherein the mCNPs are less than 100 nm, less than 50 nm, less than 25 nm, less than 15 nm or less than 10 nm in size.

9. The dispensable composition of claim 1, wherein the mCNPs are 20-24 nm in size.

10. The dispensable composition of claim 1, wherein the mCNPs are 3-5 nm in size.

11. The dispensable composition of claim 1, wherein the mCNPs comprise predominantly 3+ surface charge and a Janus-type two-phase construct.

12. The dispensable composition of claim 1, wherein the mCNPs comprise a predominantly 4+ surface charge and possess both catalase and superoxide dismutase-like enzyme-mimetic activities or where mCNPs comprise a predominantly 3+ surface charge and possess superoxide dismutase-like enzyme-mimetic activities.

13. The dispensable composition of claim 3, wherein the AgCNPs are produced via a method comprising dissolving cerium and silver precursor salts that include cerium and silver nitrates; oxidizing the dissolved cerium; and evolving the dissolved silver precursor salts to metallic silver phases on cerium surfaces.

14. The dispensable composition of claim 13, wherein the oxidizing the dissolved cerium includes oxidizing the dissolved cerium via admixture with one of: hydrogen peroxide and ammonium hydroxide.

15. (canceled)

16. A formulation for a disinfectant comprising the dispensable composition of claim 1, a drying agent, an organic acid, surfactants, a polymer binder, and water.

17. The formulation of claim 16, wherein the formulations contains one or multiple of the dispensable compositions.

18. The formulation of claim 17, wherein the metal is metallic silver (Ag) and the mCNPs of the formulation contain AgCNPs.

19. The formulation of claim 16, wherein the dispensable composition is in an amount ranging from about 0.1 -10% by weight.

20. The formulation of claim 16, wherein the drying agent is selected from the group comprising of ethanol and isopropanol.

21. The formulation of claim 20, wherein the drying agent is ethanol.

22. The formulation of claim 21, wherein the ethanol is in an amount ranging from about 0 to 40% by weight.

23. The formulation of claim 16, wherein the organic acid is citric acid.

24. The formulation of claim 23, wherein the citric acid is in an amount ranging from about 0.5 - 2% by weight.

25. The formulation of claim 16, wherein the surfactants are selected from the group comprising of lauramine oxide, myristamine oxide, other zwitterionics, tergitol 15-S-15 or other secondary alcohol ethoxylate.

26. The formulation of claim 16, wherein the surfactants are in an amount ranging from about 0.5 - 3% by weight.

27. The formulation of claim 25, wherein the surfactants are lauramine oxide and tergitol 15-S-15.

28. The formulation of claim 27, wherein the lauramine oxide is in an amount ranging from about 0.25-2% by weight.

29. The formulation of claim 27, wherein the tergitol 15-S-15 is in an amount ranging from about 0 -1% by weight.

30. The formulation of claim 16, wherein the polymer binder is selected from the group comprising of Poly(2-ethyl-2-oxazoline) and PVP-Vinyl Acetate copolymers.

31. The formulation of claim 30, wherein the polymer binder is Poly(2-ethyl-2-oxazoline).

32. The formulation of claim 31, wherein the Poly(2-ethyl-2-oxazoline) is in an amount ranging from about 1-25% by weight.

33. The formulation of claim 30, wherein the PVP-Vinyl Acetate copolymer is in an amount ranging from about 1-30% by weight.

34. The formulation of claim 16, wherein the water is in an amount ranging from about 15 - 45% by weight.

35. An apparatus comprising the formulation of claim 16; a container into which the formulation is disposed; and a nozzle for dispensing the formulation.

36. The apparatus of claim 35, further comprising a pump in communication with the formulation.

37. The apparatus of claim 35, wherein the container is fluid tight and wherein the formulation further comprises a propellant.

38. A disinfecting film comprising the formulation of claim 16 disposed on a substrate.

39. The disinfecting film of claim 38, wherein the disinfecting film can perform a residual disinfection.

40. The disinfecting film of claim 39, wherein the residual disinfection occurs in 15 to 30 minutes.

41. The disinfecting film of claim 38, wherein the disinfecting film can perform a rapid disinfection.

42. The disinfecting film of claim 41, wherein the rapid disinfection occurs in less than 1 minute.

43. The disinfecting film of claim 36, wherein the disinfecting film is active for 1 to 14 days.

44. The disinfecting film of claim 38, wherein the disinfecting film is activated upon exposure to water.

45. The disinfecting film of claim 38, wherein a dried disinfecting film can be reactivated by exposing to water.

46. A method of disinfecting a surface, the method comprises dispensing the formulation of claim 16 onto the surface.

47. A fabric comprising the composition of claim 1 disposed on a surface thereof.

48. The fabric of claim 47, wherein the fabric is configured to comprise at least a portion of a garment, mask, glove, or any other PPE.

49. A dispensable composition comprising cerium oxide nanoparticles (CNPs) and silver-associated cerium oxide nanoparticles (AgCNPs) and an excipient wherein the AgCNPs comprise a metallic silver and a fluid comprising the excipient and the AgCNPs have ionized silver removed.

50. The dispensable composition of claim 13, wherein the dissolved cerium and silver precursor salts are in a solution; and the oxidizing and precipitating the dissolved cerium and silver precursor salts include:
washing the solution;
resuspending precipitated nanoparticles of the solution; and
adding ammonium hydroxide to the resuspended solution, and
washing the residual particles.
